(19) United States
(12) Patent Application Publication
(10) Pub. No.: US 2004/0249343 A1
(43) Pub. Date: Dec. 9, 2004

(54) Combination treatment catheters and post treatment stents

(75) Inventor: Iulian Cioanta, Weston, FL (US)

Correspondence Address: BRADLEY M GANZ, PC, P O BOX 10105, PORTLAND, OR, 97296

(73) Assignee: WIT IP Corporation

(21) Appl. No.: 10/859,666

(22) Filed: Jun. 2, 2004

(30) Foreign Application Priority Data
Dec. 6, 2002 (WO) PCT/US02/38641

(51) Int. Cl. A61F 7/12

(52) U.S. Cl. 604/113; 607/113

(57) ABSTRACT

Combination catheters/stents includes matable female and male members which, when engaged, deliver the desired therapy, typically a thermal therapy, to the targeted tissue in the body. At the conclusion of the delivery of the therapy regimen, the female and male matable members disengage allowing one member (the stent portion) to remain in the body proximate the treatment site of the subject while the other member (primarily the catheter portion which with the stent portion allows delivery of desired treatment) can be removed from the body of the subject. The combination catheter/stent requires only a single insertion procedure reducing the trauma potentially introduced to tissue (either along the lumen or at the targeted treatment site) in the body.

The treatment catheters and methods are particularly suitable for the treatment of BPH. Associated methods are also described.

### RELATED APPLICATIONS

This application claims priority from U.S. Provisional Application Ser. No. 60/339,529, filed Dec. 10, 2001, and PCT Application Serial No. PCT/US02/38641 filed on Dec. 6, 2002, the contents of which are hereby incorporated by reference as if recited in full herein.

### FIELD OF THE INVENTION

The present invention relates to catheters and stents configured for insertion into a lumen or body cavity of a subject, and is particularly suitable for catheters and stents configured for insertion into the male urethra.

### BACKGROUND OF THE INVENTION

Conventionally, several types of thermal treatment systems have been proposed to treat certain pathologic conditions of the body by heating or thermally ablating targeted tissue. These thermal treatment systems have used various heating sources to generate the heat necessary to treat or ablate the targeted tissue. For example, laser, microwave, and radio-frequency (RF) energy sources have been proposed to produce the heat that is then directed to the targeted tissue in or around the selected body cavity. Thermal treatment systems have been used to thermally ablate prostate tissue as well as to thermally treat or ablate the tissue of other organs, body cavities, and/or natural lumens.

One particularly successful thermal ablation system ablates the prostate by a thermocoagulation process. This thermal ablation system employs a closed loop liquid or water-induced thermotherapy (WIT) system that heats liquid, typically water, external to the body and then directs the circulating heated water into a treatment catheter. The treatment catheter is inserted through the penile meatus and held in position in the subject prior to initiation of the treatment that, in operation, exposes localized tissue in the prostate to ablation temperatures. The treatment catheter includes an upper end portion that, in position, is anchored against the bladder neck and an inflatable treatment segment that is positioned relative to the anchored upper end portion such that it resides along the desired treatment region of the prostate. In operation, the treatment segment expands radially outward, in response to the captured heated circulating fluid traveling therethrough, to press against the targeted tissue in the prostate and to expose the tissue to increased temperatures associated with the circulating liquid, thereby thermally ablating the localized tissue at the treatment site. In addition, the pressurized contact can reduce the heat sink effect attributed to blood circulation in the body, thus enhancing the depth penetration of the heat transmitted by the inflatable treatment segment into the prostatic tissue.

As an acceptable alternative to surgery (transurethral resection of the prostate (TURP)), the use of WIT (water-induced thermotherapy) has been shown to be a successful and generally minimally invasive treatment of BPH (benign prostatic hyperplasia). Generally stated, the term “BPH” refers to a condition wherein the prostate gland enlarges and the prostatic tissue increases in density that can, unfortunately, tend to restrict or close off the urinary drainage path. This condition typically occurs in men as they age due to the physiological changes of the prostatic tissue and bladder muscles, over time. To enlarge the opening in the prostatic urethra, without requiring surgical incision and removal of tissue, the circulating hot water is directed through the treatment catheter which is inserted into the penile meatus up through the penile urethra and into the prostate as described above. The treatment segment expands with the hot water circulated therein to press the inflated treatment segment against the prostate, which then conductively heats and thermally ablates the prostatic tissue. The circulating water is typically heated to a temperature of about 60°-62° C. and the targeted tissue is thermally treated for a period of about 45 minutes to locally kill the tissue proximate the urinary drainage passage in the prostate between the bladder neck and verumontanum (where BPH manifests) and thereby enlarge the urinary passage through the prostate.

Depending on the particular application, the treatment catheter can include insulated regions on the proximal shaft portion of the catheter to protect non-targeted tissue from undue exposure to heat as the heated fluid travels in the catheter fluid circulation passages to the desired treatment region. The insulated regions have been provided by various means such as configuring the catheter with an extra layer or thickness of a material along the proximal or lower shaft portion. Other insulation means include a series of circumferentially arranged elongated channels or conduits (either filled with air or other material, or which are configured to provide increased lateral thermal resistance), which encircle the heated circulating fluid passages and provide an axial length of thermal insulation along the elongated shaft portion of the catheter as described in U.S. Pat. Nos. 5,257,977 and 5,549,559 to Eshel, and co-pending and co-assigned U.S. patent application Ser. No. 10/011,700 to Cioanta et al., entitled, Treatment Catheters with Thermally Insulated Regions, filed Nov. 13, 2001, identified by Attorney Docket No. 9149-16, and corresponding Provisional Application Ser. No. 60/248,109, the contents of which are hereby incorporated by reference as if recited in full herein. In any event, as the heated fluid travels through the fluid circulating passages, the insulation means acts to reduce the heat transferred to non-targeted treatment sites, such as along the penile meatus, penile urethra, urethral sphincter, or membranous urethra during the BPH treatment with WIT. The insulation can also protect non-targeted tissue from exposure to reduced temperatures when cooled fluid is circulated in the catheter.

In any event, subsequent to the delivery of a treatment administered to the tissue (whether radiation, surgery, hyperthermia, or thermal ablation (or other thermal) treatment), the treated tissue in the prostate undergoes a healing process. Initially, the treated or treated tissue can expand or swell due to inflammation or edema that can undesirably block or obstruct the prostatic urethra. Further, during the healing period, portions of the treated tissue can slough off and create an undesirable and unduly limited opening size. This post-ablation or post-therapy treatment opening size can be positively influenced by “molding” the treated or ablated tissue during the healing cycle to contour the tissue about a catheter or stent held thereat.

Therefore, to facilitate proper healing and to enhance the efficacy of the therapy, and particularly ablation therapy, either the treatment catheter is left in the subject for a period of time and/or a post treatment catheter or stent, such as a conventional Foley catheter, is positioned in the subject after the treatment catheter is removed. Other examples of treatment catheters or stents are described in co-pending, co-assigned U.S. patent application Ser. Nos. 09/239,312; 09/837,486; and 10/011,494 entitled METHODS FOR TREATING THE PROSTATE AND INHIBITING OBSTRUCTION OF THE PROSTATIC URETHRA USING BIODEGRADABLE STENTS identified by Attorney Docket No. 9149-20, the contents of which are hereby incorporated by reference as if recited in full herein. Other examples of treatment catheters or stents are described in co-pending, co-assigned U.S. Provisional Patent Application Ser. Nos. 60/318,556; 60/308,344; and 60/330,029, filed Oct. 17, 2001, entitled CATHETERS WITH SUCTION CAPABILITY AND RELATED METHODS AND SYSTEMS FOR OBTAINING BIOSAMPLES IN VIVO identified by Attorney Docket No. 9149-17PR, the contents of which are hereby incorporated by reference as if recited in full herein.

Conventionally, the treatment catheter itself may be left in the subject for about 24-72 hours after delivering the thermal treatment to the targeted tissue to reduce the likelihood that the treatment site will be injured by premature removal of the treatment catheter or post-treatment insertion of a stent or conventional Foley type catheter.

### SUMMARY OF THE INVENTION

The present invention provides combination treatment and post-treatment catheters or catheters with a matable and releasable stent member (which can descriptively be termed a “catheter/stent”) and related methods. It is noted that the term “stent” is used herein in certain portions of the description to indicate the portion of the assembly which is configured to remain in the body after the targeted therapy has been delivered (and after the other portion of the catheter assembly is detached or separated from the stent and removed from the body) but may be used interchangeably with the term “catheter.” In particular embodiments, the stent portion of the device can be configured so as to reside above the urinary sphincter when in position in the body so as to allow normal function of the urinary sphincter.

In operation, the present invention provides a combination catheter/stent with matable female and male members which, when engaged, deliver the desired therapy typically a thermal therapy (either a hyper or hypo type therapy), to the targeted tissue in the body. At the conclusion of the delivery of the therapy regimen, the female and male matable members disengage, allowing one member (the stent portion) to remain in the body proximate the treatment site of the subject while the other member (primarily the catheter portion which with the stent portion allows delivery of desired treatment) can be removed from the body of the subject. The combination catheter/stent requires only a single insertion procedure, reducing the trauma potentially introduced to tissue (either along the lumen or at the targeted treatment site) in the body.

In certain embodiments, the combination treatment catheter and post-treatment stent can be configured for insertion into a body cavity or lumen of a biological subject. The combination catheter/stent can include a first member having an inner cavity and a wall having an outer surface and an inner surface and a second member. The first member can include at least one outwardly expandable inflatable treatment balloon positioned to expand outwardly away from the outer surface of the first member, such that, in position, the inflated treatment balloon contacts targeted tissue of a biological subject, and at least one inlet port extending through the wall of the first member and being in fluid communication with the at least one treatment balloon.

The second member can be configured such that at least a portion of its length is sized and configured so as to be received inside the first member cavity. The second member can include an axially extending outer wall and at least one inner fluid lumen positioned therein and extending along a length thereof. The second member can also include a plurality of fixation balloons positioned to extend outwardly from the second member outer wall to releasably engage with the inner surface of the first member, when the second member is in position in the first member cavity, and a plurality of inflation ports, at least one of which extends between an inflation path in the second member and a respective one of the fixation balloons. The second member can also include an outlet port in fluid communication with the at least one inner fluid lumen and extending through the second member outer wall such that when engaged, the first and second members cooperate to provide at least one enclosed chamber extending between the second member outer wall and the first member inner wall. The at least one enclosed chamber defines a fluid passage which, in operation, allows a fluid to be directed, in serial order, from the second member inner fluid lumen through the second member outlet port, into the at least one fluid chamber, into the inlet port of the first member and to the first member treatment balloon to expand the treatment balloon responsive to the introduction of fluid therein.

In certain embodiments, the combination treatment catheter/stent can be configured for insertion into a body cavity or lumen of a biological subject, and may include a flexible elongated tubular body having a first member and a releasably attached second member. In position, the first and second members cooperate to generate and deliver a desired therapy to tissue in a localized region of the body. After the desired therapy has been delivered, the first and second members are disengaged and a selected one of the members is slidably removed from the body of the subject and the other member is left in position proximate the treated tissue.

In some embodiments, the first member is a female member and the second member is a male member configured to be received in the female member. The female member can include an inflatable treatment balloon positioned about a peripheral distal portion of the female member such that the treatment balloon is expandable to a configuration which extends radially outward a distance from the outer wall of the female member. The male member may include a plurality of fixation balloons configured to expand outwardly to secure the first and second members together.

In certain embodiments, methods of thermally treating a subject may comprise the steps of: (a) inserting a flexible combination treatment catheter/stent into a natural lumen or body cavity of a subject, the combination catheter/stent comprising an elongated male member having a first length and an elongated female member having a second length, the first length being substantially larger than the second length, wherein the male member includes at least one inflatable fixation balloon configured to expand to contact and securely attach the female and male members together such that they are operatively associated during delivery of a desired thermal treatment to the lumen or body cavity; (b) delivering a desired thermal treatment to a target site in the lumen or body cavity from the combination treatment catheteristent; (c) collapsing the at least one fixation balloon to disengage the male member from the female member after said delivering step; (d) removing the male member from the body of the subject after the delivering and collapsing steps; such that the female member remains in position in the subject.

In certain embodiments, methods of administering a therapy to a natural lumen or body cavity of a subject comprise: (a) inserting a catheter formed of a first member releasably attached to a second member into the body cavity or lumen of a subject; (b) delivering a therapy to targeted tissue in the lumen or body cavity of the subject via the catheter such that the therapy is generated by a cooperating relationship between the first and second members; (c) releasing the first and second members from each other after the delivering step; and (d) removing the second member from the body after the releasing step such that the first member remains in position in the body of the subject.

In certain embodiments, the combination catheter/stent is configured to be inserted into a body cavity or natural lumen and includes an outer stent member which has, a cavity and an inner treatment catheter member which has an elongated body, a portion of which is configured to reside inside the stent member cavity and to releasably engage with the stent member. When engaged, the outer stent member and the inner treatment catheter member are configured to cooperate to deliver a desired thermal treatment to the targeted region. Subsequently, the inner treatment catheter can be released from the outer stent member and removed from the subject, leaving the stent member in position in the localized treatment region in the body of the subject to help contour the healing tissue and/or to inhibit the closure of the lumen or cavity.

In some embodiments, the treatment catheters can be provided as a set of prostatic treatment catheters, each configured for insertion into the male urethra (such as for treating BPH). However, the set is provided such that each treatment balloon which expands to deliver the thermal treatment is sized a different length to allow customized fit to a particular subject (the treatment balloon is adapted to reside about the prostatic urethra, the length of which can vary from patient to patient, and the catheter can be configured to accommodate this variability by sizing the catheter treatment balloon itself in a range of lengths from about 2-6 cm, typically in increments of about ½ cm).

Certain of the embodiments described are particularly suitable for a subject undergoing thermal therapy or thermal ablation treatment to a localized target region in a natural body cavity or lumen such as within the prostatic urethra. The treatment catheter portion can be conveniently removed during the treatment session (typically after the desired thermal therapy has been completed). Removal of the catheter portion can be accomplished by the clinician sliding the treatment catheter portion from the body while leaving the stent member in place. This process can eliminate the requirement of a post-therapy insertion (which may reduce one or more of bleeding, clotting, cramping, and additional tissue damage) while allowing for the use of a more comfortably configured stent that can inhibit the lumen or cavity from closing down.

In certain embodiments, in lieu of or with the thermal treatment, the combination catheter/stent can be used to deliver medicaments, rinses, fluids, supplements, radiation therapy, gene therapy or other therapy types to the targeted treatment region. The combination catheter/stent can include one or a combination of suitable coatings such as hydrophilic coatings that can help the ease of insertion into the body cavity, as well as one or more of antimicrobial coatings, anti-inflammatory coatings, anti-scarring coatings, and antibiotic coatings.

Particular embodiments of the present invention are directed to methods of making a combination treatment catheter/stent device configured for insertion into a body cavity or lumen of a biological subject. The methods include: (a) configuring a flexible elongated tubular body using a first member and a releasably attached second member, wherein, when attached and in position in a subject, the first and second members cooperate to generate and deliver a desired therapy to tissue in a localized region of the body of the subject, and wherein after the desired therapy has been delivered, the first and second members are disengagable from each other while they are held in vivo and a selected one of the members is slidably removable from the body of the subject while the other member remains in position proximate the treated tissue.

The first member can be formed with an inner cavity and an outwardly expandable thermal treatment balloon; and the second member can be formed so that it is sized and configured to be received within the first member cavity and provides at least one fluid flow channel, so that when properly attached together, the first and second members are oriented to be in cooperating alignment so as to be able to transfer liquid from the fluid flow channel in the second member to the treatment balloon on the first member to deliver a thermal therapy.

In certain embodiments, the second member is configured to be removable from the first member and is independently removeable from the subject while the first member is adapted to remain in the subject as a post-treatment stent held proximate the operative treatment location for a desired post-treatment duration. In addition, the method can include attaching a conduit having a cross-sectional area that is substantially less than that the cross-sectional area of the first and/or second member to a selected one of the first and second members, the selected member being the member adapted to remain in the subject after the other member is removed. Further, the method may include forming a plurality of laterally expandable fixation balloons on at least one of the first and second members so as to releaseably secure the first and second members theretogether. In particular embodiments, the forming step can be carried out so that the fixation balloons also provide a plurality of enclosed fluid flow channels that extend between the first and second members when the first and second members are attached together.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1A is a side sectional view of a stent member according to embodiments of the present invention.

FIG. 1B is a partial side sectional view of an elongated treatment catheter member according to embodiments of the present invention.

FIG. 2A is a partial side sectional view illustrating the treatment catheter member of FIG. 1B as it is positioned to be inserted into the stent member of FIG. 1A according to embodiments of the present invention.

FIG. 2B is a partial side sectional view illustrating the assembly of members of FIG. 2A with the two members coupled together and securely attached to each other according to embodiments of the present invention.

FIG. 3 is a partial side sectional view of the assembly of FIG. 2B shown in an operative configuration according to embodiments of the present invention.

FIG. 4 is a cross-sectional view taken along line 44 in FIG. 3.

FIG. 5 is a cross-sectional view taken along line 5-5 in FIG. 3.

FIG. 6 is a cross-sectional view taken along lines 6-6 in FIG. 3.

FIGS. 7A and 7B are partial axial section views of the catheter/stent assembly according to embodiments of the present invention, FIG. 7B being rotated 90 degrees from the orientation shown in FIG. 7A.

FIGS. 8-15 are schematic section views of the catheter/stent assembly of FIGS. 6 and 7A, 7B, illustrating a sequence of operational configurations as the device is positioned and operated in a subject according to embodiments of the present invention. FIG. 8 illustrates a configuration at insertion. FIG. 9 illustrates a distal balloon expanded to locate the device in an operative position in the body of the subject. FIG. 10 illustrates the configuration during active administration of a thermal therapy. FIG. 11 illustrates the configuration at the end of the active thermal therapy session. FIGS. 12 and 13 illustrate the configuration of the stent member of the device as anchors are engaged to help locate the stent member in the body apart from the catheter so that it can remain in the body after the other catheter portion is removed. FIG. 14 illustrates the detachment of the two members from each other and the deflation or collapse of the distal balloon. FIG. 15 illustrates the elongated member being removed from the body leaving the stent member in position.

FIG. 16 is a side sectional view of an alternate embodiment of the present invention.

FIG. 17 is a partial side sectional view of an additional embodiment of the present invention.

FIG. 18 is a partial side sectional view of yet another embodiment of the present invention.

FIG. 19 is a partial side sectional view of still another embodiment of the present invention.

FIG. 20 is a cross-sectional view taken along line 20-20 in FIG. 19.

FIG. 21 is a partial cutaway view of an alternate embodiment of the treatment catheter member shown in FIG. 19.

FIG. 22 is a cross-sectional view taken along line 22-22 in FIG. 21.

FIG. 23 is a block diagram of a method for delivering a therapy to a subject according to embodiments of the present invention.

FIG. 24 is a block diagram of a method of delivering a thermal therapy to a subject according to embodiments of the present invention.

FIG. 25 is a block diagram of a method of delivering a thermal therapy to the prostatic urethra according to embodiments of the present invention.

### DETAILED DESCRIPTION OF EMBODIMENTS OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. In the figures, certain elements, regions, or features may be exaggerated for clarity. Like numbers refer to like elements throughout.

For ease of discussion and clarity, the combination treatment catheter/stent will primarily be described below as it relates to thermal treatments delivered via circulating fluid systems for prostate applications. However, the combination catheter/stent devices of the present invention may also be configured for insertion in other natural lumens or body cavities such as, but not limited to, blood vessels including arteries, the colon, the uterus, the cervix, the throat, respiratory passages, the ear, the nose, and the like.

In addition, it is noted that the combination treatment catheter/stents of the present invention can be configured to deliver therapies to targeted tissue in natural lumens or body cavities other than, or as a supplement to, thermal therapies (whether cooling and/or heating), such as, but not limited to, drug therapies, gene therapies, radioactive seeds or cancer therapies. For example, the combination catheter can be used to deliver bioactive, bio-reactive, and/or therapeutic agents to targeted tissue in the body.

In certain embodiments, the combination device can be configured to transfer an exogenously introduced fluid to provide the desired treatment. The exogenous fluid can be a heated liquid that expands a treatment balloon or a liquid or pharmaceutical agent that is expelled or applied by the device.

For heat-based thermal treatment configurations, various heating techniques may be employed to generate the heat necessary to treat or ablate the targeted tissue. For example, laser, microwave, ultrasound, circulating liquid, and radio-frequency (RF) energy sources have been proposed to produce the heat which is directed to the targeted tissue in or around the selected body cavity or lumen. Thus, the heating source can be any suitable heating source but is primarily described herein as a circulating liquid heating system for ease of description and for illustrative purposes and is not meant to be limiting to the various heating types which may be employed (and the heating element or source may be located on or in the catheter/stent or located external of the body).

In certain embodiments related to thermal ablation therapies, the targeted tissue is exposed to an elevated temperature that is greater than or equal to about 45° C. for a predetermined period of time. The treatment catheters/stents of the present invention may also be used for other thermal therapies such as to deliver cooled liquids (cooled to temperatures below the average body temperature such as to about 15-2° C. or even to 0° C. and/or cryogenic temperatures) or to deliver heated liquids heated to temperatures below 45° C.) to a target region in the cavity or natural lumen in the subject's body. Thus, the term “thermal therapy” applies both to cooling and heating therapies. The present invention finds use for both veterinary and medical applications. Treatment subjects, according to the present invention, include animal subjects, and are preferably mammalian subjects (e.g., humans, canines, felines, bovines, caprines, ovines, equines, rodents, porcines, and/or lagomorphs), and more preferably are human subjects.

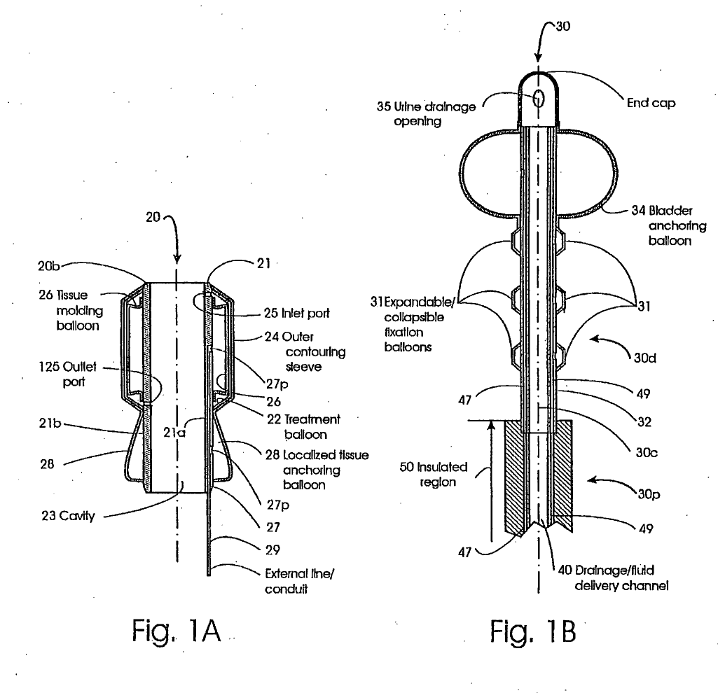

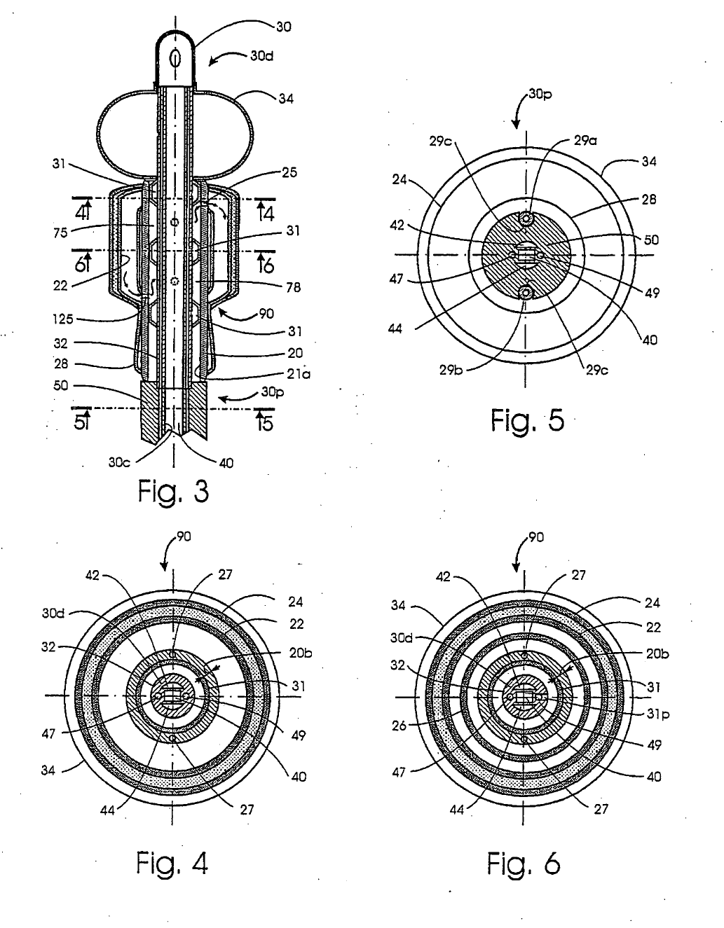

Turning now to FIG. 1A, one embodiment of a first member that can be descriptively termed “a stent member” 20 is shown. As shown, the stent member 20 includes a wall 21 and an inflatable or expandable treatment balloon 22 positioned to expand outwardly away from the wall 21. The stent member 20 is configured to define a cavity 23. The stent member 20 may, in some embodiments, include a contouring sleeve 24 overlying the treatment balloon 22 and/or a tissue-molding balloon 26 underlying the treatment balloon 22. The stent member 20 can be formed of an elastomeric material and is configured to be sufficiently rigid so as to retain its shape independent of the underlying second member (30, FIG. 3). As shown in FIG. 3, the wall of the stent member 20 is sized to be radially spaced-apart from the wall of the inner member with a radially extending gap space therebetween.

Referring again to FIG. 1A, as shown, the expandable treatment balloon 22 is intermediate the contouring sleeve 24 and the tissue-molding balloon 26. The tissue-molding balloon 26 may be configured to be separately inflatable from the treatment balloon 22. The tissue-molding balloon 26 shown resides between the treatment balloon 22 and the wall 21 and has a shorter length than the treatment balloon 22. As shown, in some embodiments, this can allow the treatment balloon 22 to expand without forcing the tissue-molding balloon 26 to also expand. In contrast (as shown in FIG. 15), when the tissue-molding balloon 26 expands outwardly away from the wall 21 of the stent member 20, the treatment balloon 22 and sleeve 24 reside against the underlying tissue molding balloon 26 and they concurrently expand as the tissue molding balloon 26 forces them outward.

As shown by the shading in FIG. 3, a fluid (such as a non-toxic liquid) may be interposed between the sleeve 24 and the treatment balloon 22 to enhance thermal transmissivity therebetween or so as to allow the fluid to migrate through the sleeve (that may be permeable) to therapeutically treat the local tissue. The treatment balloon 22 may be formed of a different material than the contouring sleeve (such as PVC for the treatment balloon and silicon for the contouring sleeve). Additional discussion of suitable contouring sleeves 24 can be found in co-pending, co-assigned U.S. Provisional Patent Application Ser. No. 60/288,774, entitled, Low Thermal Resistance Elastic Sleeves for Medical Device Balloons, filed May 4, 2001, the contents of which are hereby incorporated by reference as if recited in full herein.

FIG. 1A also shows a localized tissue anchoring balloon 28 mounted to the wall 21 of the stent member 20 such that it is spaced apart from the treatment balloon 22 and tissue molding balloon 26. The stent member 20 can also include one or more elongated conduits 29 extending from one end and an inflation path 27 extending in the wall 21 of the stent member 20. Alternatively, the conduit 29 can be directed to thread through the wall 21 (or be held contoured into, or adjacent along the inner or outer surface 21a, 21b thereof, respectively) to provide one or more inflation paths to one or more of a desired balloon 22, 24, 26, or 28. The stent body 20b can be configured with spaced apart tubular walls so that the gap between the walls form part of the inflation path (not shown).

As shown in FIG. 1A, the inflation path 27 extends from the conduit 29 and into and axially along a length of the wall 21 to inflation ports 27p operably associated with the tissue molding balloon 26 and the localized anchoring balloon 28. Separate inflation paths 27 and/or conduits 29 may also be used to inflate the balloons 28, 26 (so as to either be in fluid isolation or in fluid communication and/or to be either concurrently or separately inflatable). As shown in FIG. 1A, the conduit 29 may be directly connected with the inflation path 27 formed in or defined by the tubular wall(s) 21 of the stent body 20b through which the inflation medium is directed. Suitable inflation media include gas, liquids, or solids/powders or mixtures thereof including, but not limited to, air, noble gases such as nitrogen and helium, oxygen, water, and oils (such as canola oil, olive oil, and the like). Preferably, the inflation medium is selected to be non-toxic and to reduce any noxious effect to the subject should the balloon integrity be compromised, accidentally rupture, leak, or otherwise become impaired during service. In certain embodiments, a liquid (or a substantially liquid media) be used to inflate at least the tissue anchoring balloon 28 to extend the time that the balloon 28 will remain substantially inflated during chronic (during the post-treatment healing process) positioning in the body. Due to the thin wall of the inflatable balloon, air or gas may more easily migrate from the balloon thereby allowing the balloon to deflate prematurely or to become more compressible (and potentially less effective to anchor in the desired location) as it releases or loses inflation media.

As shown in FIG. 1A, in some embodiments, the stent member 20 may include at least one inlet port 25 extending through the thickness of the wall 21 to provide a fluid flow path from the cavity 23 to the treatment balloon 22. Similarly, the stent member 20 may include an outlet port 125 which can be circumferentially and axially spaced apart from the inlet port 25. The conduit 29 has a cross-sectional width which is substantially smaller than that of the stent member 20 itself and may have a length sufficient to extend out of the subject's body when in position therein. In one embodiment, a plurality of conduits 29 separately extend about the circumference of the body of the stent member 20. Where, as in some applications, inflatable tissue molding balloons or tissue anchoring balloons are not employed, the conduit may be a string, thread, line, or other element which can be accessed and pulled to remove the stent member 20. As shown in FIG. 1A, the conduit 29 and the inflation path 27 in the wall direct an inflation medium to inflate both the tissue molding and anchoring balloons 26, 28.

It is also noted that other stent anchor configurations may also be used as well as other stent body configurations, typically depending on the particular application. For example, the stent body may have a longer length with a transurethral bridge configuration (two members separated by a collapsible or substantially reduced intermediate portion that allows normal functioning of the urinary sphincter) or may employ frictional tissue engagement members or spring members to help locate and/or hold the stent in the body. See e.g., U.S. Pat. Nos. 5,916,195, 5,766,209, and 5,876,417, the contents of which are hereby incorporated by reference as if recited in full herein.

As shown in FIG. 15, in certain embodiments, the stent 20 is a unitary body 20b that has an axial length such that, when in position in the subject, it extends above the sphincter 13 through the prostatic urethra. As shown, the stent conduit or tube 29 has a length sufficient to extend from the stent body 20b to a position that is external of the subject when the stent 20 is in position in the prostatic cavity. The conduit 29 is configured with a shape and/or cross-sectional size, which is substantially smaller than the stent body cross-sectional size or width such that it is sufficiently small to allow normal function of the sphincter 13. The localized tissue-anchoring balloon 28 is in fluid communication with the conduit or tube 29 and an external inflation source. As such, the stent 20 is sized and configured to reside in the subject above the sphincter 13. In other embodiments, alternately shaped or longer stent bodies may be desired.

In this embodiment, unlike incontinence catheters or transurethral bridge stent configurations, the stent body 20b may be configured to reside entirely above the sphincter 13 so that only one or more substantially smaller diameter (or cross-section) tube(s) 29 extend below the subject's sphincter to exit the penile meatus. As only one or more tubes 29 extend through the sphincter 13, the stent 20 configuration allows natural operation of the sphincter 13 (i.e., the sphincter can close substantially normally with the stent 20 in position) thereby reducing the complexity and invasiveness of the device. The width or outer diameter of the stent body 20b may be about 6-9 mm and the conduit 29 may be sized to be at least about 20-25 percent less than the cross-sectional width or outer diameter of the stent body 20b. In some embodiments, the conduit(s) 29 has an outer cross-sectional width or diameter which is from about 1 mm-2.25 mm.

In operation, in order to anchor the stent 20 in a desired position or location within the targeted lumen or cavity, (such as after the treatment therapy when the stent 20 is in position in the prostate 11) the stent localized tissue anchoring balloon 28 or inflatable segment is inflated via a fluid introduced through the conduit 29 to an expanded configuration. The tissue-molding balloon 26 may be inflated before, after, or concurrently with the inflation of the localized anchoring balloon 28. When expanded, the tissue-molding balloon 26 and localized anchoring balloon 28 are adapted to expand to engage with adjacent tissue (when deflated, the balloons substantially collapse against the stent body 20b to present a relatively smooth substantially constant profile to allow for ease of insertion and removal into and out of the body).

In certain embodiments, as shown in FIG. 15, the stent 20 may be configured such that the tissue anchoring balloon 28 engages with urethral tissue which is below the treatment region 10 but above the sphincter 13, and preferably in the membranous urethra. As such, in certain embodiments the localized anchoring balloon 28 is configured to expand outwardly the greatest distance at a location between the verumontanum 11b and sphincter 13. The tissue-molding balloon 26 is configured to expand outwardly the greatest distance at a location above the verumontanum and with an axial length that extends for a major portion of the distance between the bladder neck and verumontanum 11b. In position, the localized anchoring balloon 28 engages with tissue located above the sphincter 13 and below the verumontanum 11b to help locate and secure the stent member 20 in position in the body. The tissue-molding balloon 26 can expand to engage with treated tissue to help mold the shape or inhibit closure of the lumen or cavity proximate the treated area (as this region can experience inflammation and swelling due to the treatment). FIG. 15 also shows the conduit 29 relative to the sphincter 13 illustrating the inward movement of the conduit(s) relative to the stent body 20b, when in position, allowing the sphincter 13 to function substantially normally when the stent 20 is proper position in situ and the catheter member 30 is removed.

In some embodiments, the tissue-anchoring balloon 28 may be configured to take on an inflated shape which can be described as a pear shape, ramped or inclined shape (increasing size from top to bottom), or frustoconical shape. This allows the profile of the tissue-anchoring balloon 28 to taper out from the top to the bottom, thereby inhibiting movement of the stent 20 toward the sphincter 13 when the sphincter 13 relaxes or opens. In addition, this shape may also inhibit upward movement of the stent body 20b toward the treatment region 10 or bladder 12, as the upper portion of the prostatic urethra, especially when the treated tissue is swollen, inflamed or suffering from edema, tends to close down or restrict the opening area in this region. Thus, the tapered anchoring balloon 28 may be positioned in the membranous urethra such that it abuts the restricted size of the urethral canal thereabove, in the treatment region, thereby inhibiting upward movement or migration of the stent 20. Of course, the present invention is not limited thereto and other localized balloon shapes may also be employed such as bulbous, elliptical, oval, cylindrical, accordion pleated, tapered from (such as circumferentially disposed about the perimeter of the lower portion of the stent body), and the like.

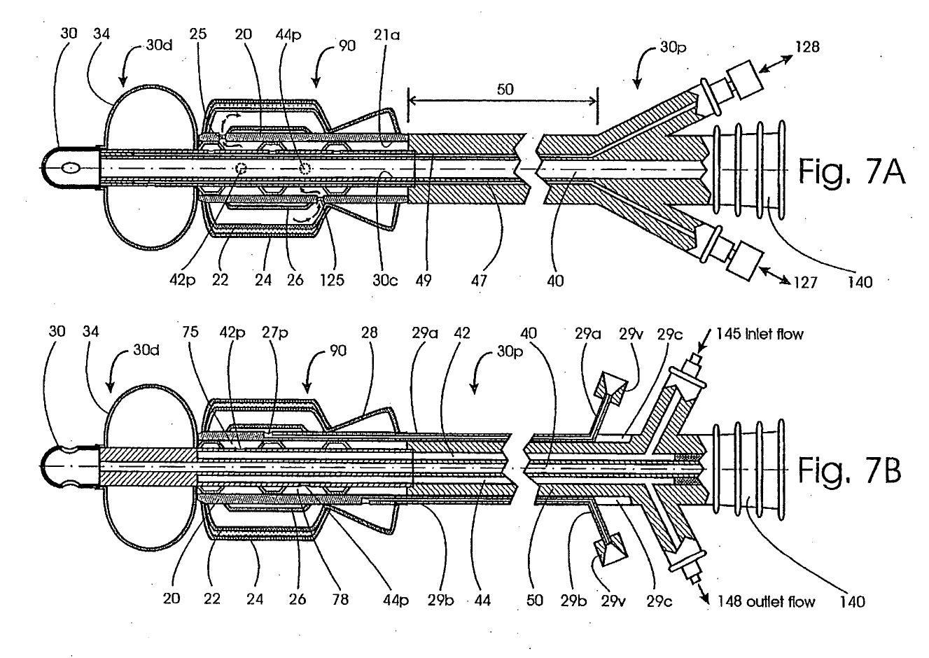

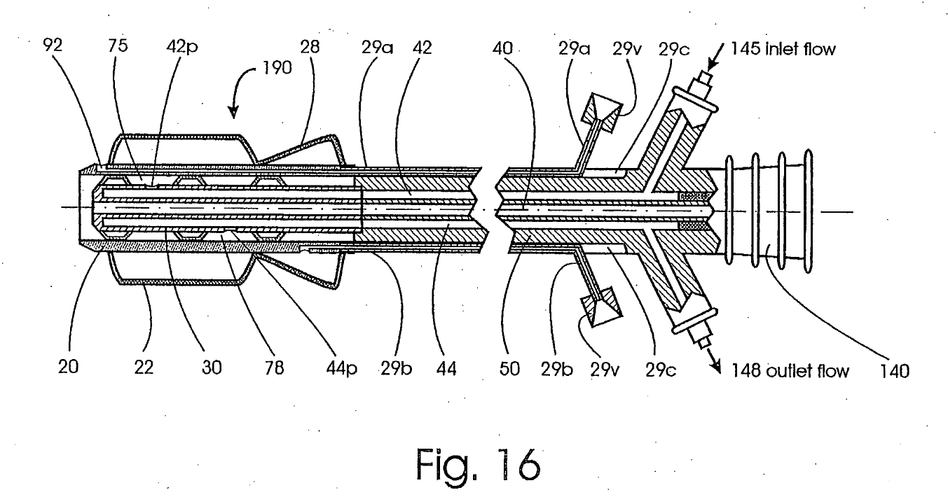

As noted above, the tissue molding and tissue anchoring balloons 26,28, respectively, may be in fluid communication with at least one common or separate conduit 29. In certain embodiments, the conduit 29 can be operatively associated with a valve 29V (FIG. 7B) and a fluid inflation source. Suitable valves are well known to those of skill in the art and are available from medical suppliers or manufacturers such as Alaris Medical Systems of Creedmoor, N.C. and San Diego, Calif., Halkey-Roberts of St. Petersburg, Fla., and B. Braun of Bethlehem, Pa. In operation, inflation media (liquid, gas, or a mixture of one or more of liquid, gas and/or a powder or solid (which may dissolve after exposure to the gas or liquid)) is directed into the conduit or tube 29 and up into the tissue anchoring balloon 28 (or other desired region such as the tissue molding balloon 26, or medicine or fluid delivery port 92 (FIG. 16)).

FIG. 16 illustrates that the stent member 20 can include two (or more) conduits 29a, 29b, one in fluid communication with the inflatable localized tissue anchoring balloon 28 and one in fluid communication with a medication delivery port 92. Medication, drugs, treatments, rinses, and the like can be introduced into the subject into the conduit 29b such that the fluid (or fluid mixture) is then directed through the stent member 20 to exit at the delivery port 92. In one embodiment, the medication port 92 is operably associated with a distribution channel (not shown) which is formed into the outer surface of the stent member 20 such that it extends circumferentially (90-360 degrees) around the perimeter of the stent body 20b so as to allow the fluid to flow therein to facilitate a broader dispersion of the released fluid. Alternatively, in certain embodiments, a plurality of delivery ports 92 can be positioned about the perimeter to disperse the fluid. The medication can be introduced into the conduit via an externally located inlet port (not shown) which can be provided by any suitable valve/port device as is known to those of skill in the art. Suitable valve devices from Halkey-Roberts and B. Braun as noted above. The medication can be used to reduce edema, inhibit bacterial infections, reduce inflammation or to inhibit or treat the onset of urinary tract infection (UTI) or otherwise promote healing and/or treatment.

As shown in FIGS. 5, 7B, and 16, the outer wall of the treatment catheter 30 may include a groove, a recess or contour 29c sized to receive the conduit(s) 29 therein so as to hold the conduit to be substantially flush with the outer profile of the body of the catheter. Upon removal of the catheter member 30, the conduit(s) 29 simply slides along the recess 29c until they release upon the disengagement of the catheter member 30 from the stent member 20 (see e.g., FIG. 15).

In certain embodiments, as shown in FIG. 1A, the inflatable tissue molding portion 26 can be held on the stent body 20b such that it is above the tissue anchoring portion 28 (in other embodiments, no tissue molding balloon is used or it can be located below the localized tissue anchoring balloon). As shown in FIG. 15, the inflatable tissue molding balloon 26 can be configured to extend proximate the treatment region 10 when the stent 20 is in position in the subject. The tissue-molding balloon 26 may be substantially cylindrical when expanded to help form or mold the opening in the treated region (such as the prostatic urethra) to a width or outer diameter commensurate therewith as the ablated tissue heals, so as to promote an increased opening size, potentially prolonging the success of the treatment. In certain embodiments, the inflatable tissue molding balloon is sized such that when inflated it presents an outer diameter or width of about 15-25 mm. Other embodiments may employ lesser or greater dimensions.

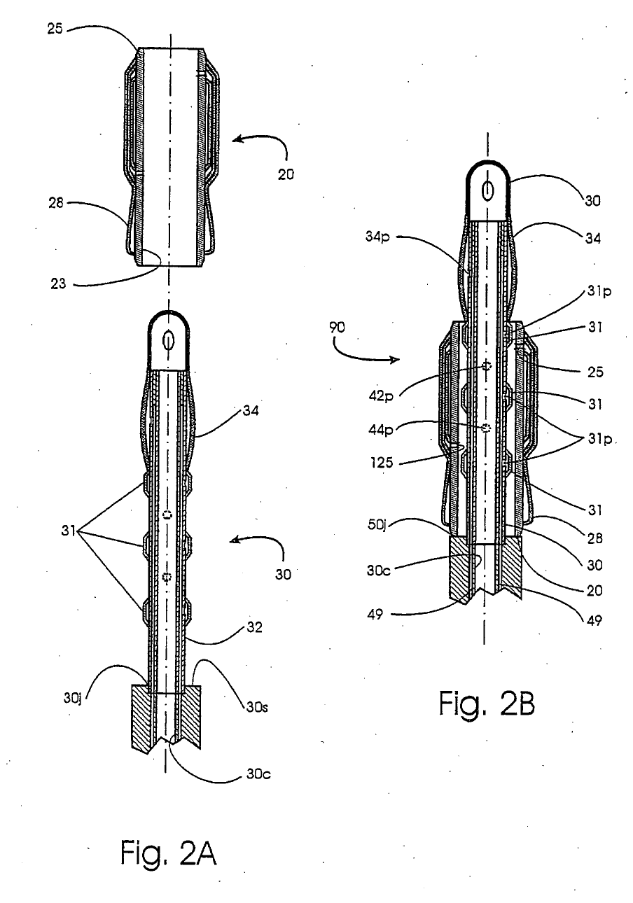

FIG. 1B illustrates one embodiment of a second member 30 which can be termed the “treatment catheter member” 30. As shown in FIGS. 2A and 2B, a portion of the treatment catheter member 30 is configured to reside in the cavity 23 of the stent member 20. FIG. 2A illustrates the treatment catheter member 30 as it is aligned with and configured to be inserted into the stent member 20. FIG. 2B illustrates the configuration of the treatment catheter member 30 as it is held securely affixed or attached to the stent member 20. As shown, a plurality of fixation balloons 31 that are in a collapsed configuration in FIG. 2A and in an expanded configuration in FIG. 2B as will be discussed further below.

Turning again to FIG. 1B, the treatment catheter member 30 shown includes a distal portion 30d and a more proximal portion 30p. In the embodiment shown, the distal portion 30d includes a plurality of spaced apart fixation balloons 31 attached to the outer surface of the wall of the member 30, an upper (or distal) anchoring balloon 34 and a fluid drainage and delivery port 35 formed into a closed distal end of the treatment catheter member (but which may be provided by an open end). As shown, the fluid drainage and delivery port 35 is in fluid communication with an inner drainage lumen 40 extending through the treatment catheter member 30. Of course, other embodiments may omit the drainage lumen 40.

To position the assembled combination catheter/stent 90 in the body, guides or pushers can be introduced into the cavity 30c of the treatment catheter and used to insert and position the combination catheter/stent in the body cavity or lumen of a subject. For example, guide wire or style placement systems are well known. Guide wires are typically used with a catheter having an open end such as shown in FIG. 16, while stylets are used with closed end or tips (such as shown in FIG. 1B) to inhibit the stylet from contacting body tissue and potentially causing injury thereto. Other guides include inflatable attachment or fixation means which laterally expand to hold the guide to inner wall of the stent 20 until the stent is in the desired location. See U.S. Pat. No. 5,916,195 and co-assigned U.S. patent application Ser. No. 09/239,312, the contents of which are hereby incorporated by reference as if recited in full herein. In addition, although the closed end configurations of the catheter/stent 90 shown herein have been illustrated as substantially upright, they can also be curved into other configurations such as Coude or Tiemen as known to those of skill in the art.

As shown in FIGS. 9-13, in certain embodiments, the upper (or distal) anchoring balloon 34 is a bladder-anchoring balloon that is configured to reside against the bladder neck of the subject, thereby securely positioning the catheter 30 and the stent 20 in the prostate relative to the bladder 12. As the inflated or expanded balloon 34 resides against the bladder neck, movement toward the sphincter 13 is inhibited. Similarly, the tissue-anchoring balloon 28 located on the other opposing end portion of the stent 20 inhibits movement toward the bladder and toward the sphincter (thus providing bilateral anchoring in the prostate).

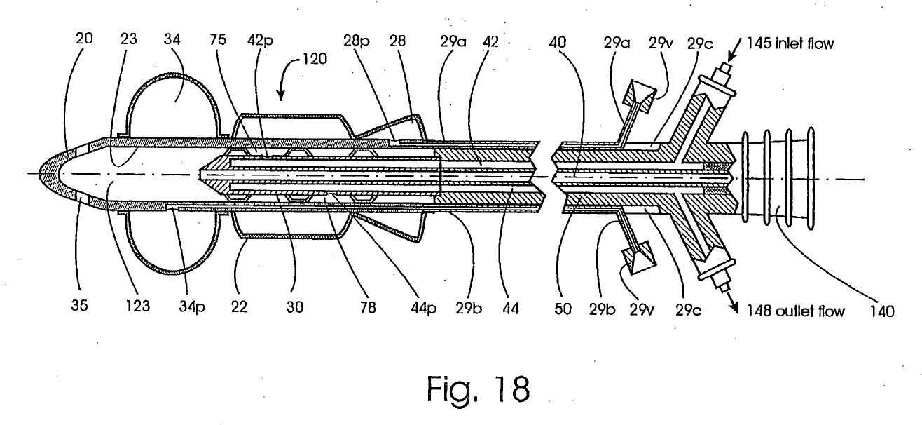

The upper anchoring balloon 34 may be separately inflatable from the fixation balloons 31 so as to allow this balloon 34 to be inflated after it is in position in the body. For the embodiment shown in FIG. 18, the stent member 120 is configured with the distal anchoring balloon 34 in lieu of the treatment catheter member 30. In this embodiment, the anchoring balloon 34 may be configured to be separately inflatable from the treatment balloon 22 (or the localized tissue anchoring balloon 28, where used). This can facilitate proper positioning of the stent member 20 in the desired region of the body, such as in the prostate relative to the bladder and above the sphincter 13.

There are three fixation balloons 31 shown in FIG. 1B, however, greater or lesser numbers may be employed. The fixation balloons 31 can be continuous about the perimeter of the outer surface (shown as circumferentially continuous about a tubular body). Upon assembly, the fixation balloons 31 are configured to expand outwardly to abut against and tightly engage with the inner surface of the wall 21a of the stent member 20 so as to be able to hold the stent member 20 and catheter member 30 fixed to each other during insertion into the body and during delivery of a desired therapy. FIG. 1B illustrates the fixation balloons 31 in an expanded configuration. FIG. 2A illustrates the fixation balloons 31 in a collapsed configuration. It is noted that, in some embodiments, the fixation balloons 31 may be alternatively or additionally positioned on the stent member 20 so as to expand inwardly to contact and hold the outer surface 32 of the distal portion of the treatment catheter member 30 (not shown).

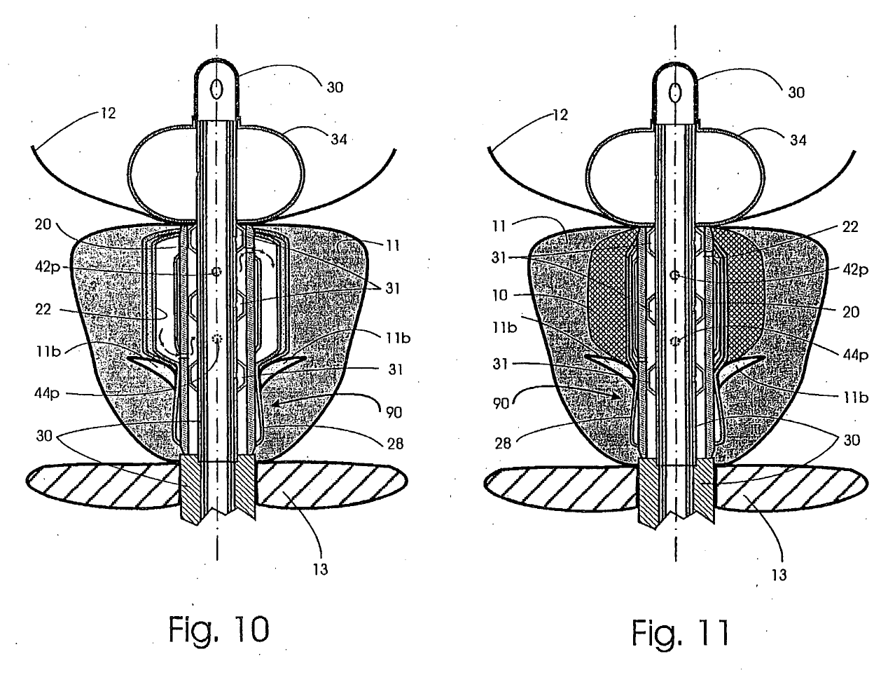

Turning to FIG. 5, this embodiment illustrates a configuration of a catheter 30 having at least three separate inner fluid lumens, which may direct liquids into or out of the body, the drainage lumen 40 (discussed above) and an inlet and outlet circulating fluid lumen 42, 44, respectively (FIGS. 4, 5, 6). In the embodiment shown in FIG. 5, the drainage lumen 40 is centrally located while the inlet and outlet circulating lumens 42, 44 are positioned on opposing sides thereof with the associated inlet port 42p being closer to the distal end than the outlet port 44p (FIGS. 7B, 10). Other lumen configurations may be employed (with different shapes and/or numbers). See e.g., co-pending, co-assigned, U.S. patent Ser. No. 10/011,700, filed Nov. 13, 2001, identified by Attorney Docket No. 9149-16, the contents of which are incorporated by reference as if recited in full herein.

Depending on the particular application, the treatment catheter member 30 can include regions having increased insulation along the length thereof (on the more proximal shaft portion of the catheter 30p) to protect non-targeted tissue from undue exposure to heat (or cooling) as the heated (or cooled) fluid travels in the catheter fluid circulation passages to the desired treatment region. As shown in FIG. 1B, in certain embodiments, the more proximal portion 30p of the treatment catheter member 30 can include an increased insulation region 50 encasing a portion of the length of the underlying lumens 42, 40, 44 (FIGS. 4, 5, 6). Various means for providing the increased insulation may be employed. For example, the insulated region 50 can be provided by configuring the outer wall of the catheter member 30 with an extra layer or thickness of a material along the proximal or lower shaft portion. Other exemplary insulation structures include a series of circumferentially arranged elongated channels or conduits (either filled with air or other material, or which are configured in appropriate geometries to provide lateral thermal resistance), which encircle the heated circulating fluid passages and provide thermal insulation along the elongated shaft portion of the catheter as described in U.S. Pat. Nos. 5,257,977 and 5,549,559 to Eshel, and co-pending and co-assigned U.S. Pat. No. 10/011,700 identified by Attorney Docket No. 9149-16 as described above (corresponding to Provisional Patent Application Ser. No. 60/248,109).

In certain embodiments, as shown in FIGS. 5, 6, and 7, the treatment catheter member 30 includes at least three separate fluid channels as noted above, the circulating inlet and outlet channels 42, 44 and the fluid drainage or medicament delivery channel 40 encased by the increased insulation region 50. In any event, as shown in FIG. 3, in certain embodiments, as heated fluid is introduced into the treatment catheter member 30 and travels through the fluid circulating passages 42, 44 (FIGS. 4, 5, 6), the increased insulation region 50 acts to reduce the heat transferred to non-targeted treatment sites, such as along the penile meatus, urethral mucosa, or urethral sphincter, during the BPH application.

As shown in FIG. 5, the treatment catheter member 30 may also include one or more separate inflation passages 47, 49 (see also FIG. 1B) which extend through the length of the treatment catheter member 30 to inflate a respective one of the distal anchoring balloon 34 and/or the fixation balloons 31. As shown in FIGS. 2B and 6, the fixation balloons 31 are in fluid communication with an inflation port 31p formed to extend from the inflation passage 49 through the wall so as to allow the fixation balloons 31 to expand and collapse responsive to inflation media being directed from an external source through the inflation passage 49 and out of the inflation ports 31p into the corresponding fixation balloon 31. Similarly, as shown in FIG. 2B, the other inflation passage 47 can be used to independently inflate and collapse the distal anchoring balloon 34 via a corresponding inflation port 34p. The number of inflation passages 47, 49 can vary depending on the application.

As shown by FIGS. 1B, 3, 4, 5, 6, the channels 47 and 49 are configured in the two portions 30p and 30d of the catheter so that they laterally and axially align between the respective portions and are in continuous fluid communication. As shown in FIGS. 5 and 6, the width or diameter of the catheter portion varies from top to bottom, the bottom having an increased width relative to the top, but the inner lumens 42, 44, 47, 49, and 40 remain substantially constant in size and location therebetween.

Turning to FIG. 2A, the treatment catheter member 30 is shown with the balloons 31, 34 collapsed as it is inserted into the stent member 20. The treatment catheter 30 includes a T-junction region 30j which includes a stop surface 30s to inhibit further forward movement into the stent member 20 to facilitate proper alignment therewith. As shown in FIG. 2B, in certain embodiments, the stop surface 30s resides against or abuts the proximal portion of the stent member 20 to close the stent cavity 23 and join the two members 20, 30 together at a common junction 50j. In certain embodiments, seals, O-rings or other components may be used (either on the mating stent surface or, and more typically, on the catheter member stop surface) to help seal the two members 20, 30 together in a manner that resists fluid exiting therefrom (not shown).

As shown in FIG. 3, in some embodiments, the stent member 20, the treatment catheter member 30, and the fixation balloons 31 define at least one enclosed chamber 75 extending between the outer wall 32 of the treatment catheter member 30 and the inner wall 21a of the stent member 20. The enclosed chamber 75 provides an enclosed (restricted) fluid travel path (shown by the lines with arrows pointing into the treatment balloon 22) between a port 42p (FIG. 2B) associated with the inlet circulating fluid channel 42 in the treatment catheter member 30 and the inlet port 25 to the inflatable treatment balloon 22 on the stent member 20 thereby causing the treatment balloon to expand responsive as fluid is directed therein.

In certain embodiments, a plurality of separate chambers (two of which can be in fluid isolation) can be employed. For example, as shown in FIGS. 3 and 7B, a second chamber 78 spaced apart from the first chamber 75 can be used to capture fluid as it exits the treatment balloon 22 via the exit port 125 on the stent member 20. The fluid then travels (shown by the dashed line arrow oriented inward) into the chamber 78 and into a return (or inlet) port 44p (FIGS. 2B, 7B) proximately located to the chamber 78 into an axially extending return channel (such as the drainage lumen 40 or the circulating fluid exit lumen 44) to travel out of the subject.

FIGS. 7A and 7B illustrate certain embodiments of the catheter/stent 90. As shown in FIGS. 7A and 7B, the proximal portion of the treatment catheter member 30p includes multiple fluid inlet and outlet ports 127, 128, 140. The two-way port 127 is in fluid communication with inflation path 47 and the two way port 128 is in fluid communication with inflation path 49 (a valve can allow the direction of fluid flow to inflate or collapse the desired balloons). The drainage lumen 40 can exit into an exit port 140 which may be in fluid communication with a drainage basin, pouch, bag, or the like. As shown in FIG. 7B, in certain embodiments, a fluid inlet port 145 can be used to direct thermally treated fluid through the inlet lumen 42, into chamber 75, out of port 42p, into port 25, and into the treatment balloon 22 as discussed above. Similarly, a fluid outlet port 148 can be used to direct captured fluid directed out of the treatment balloon 22 through port 125 into the chamber 78, into port 44p and into the exit path 44 out of the body.

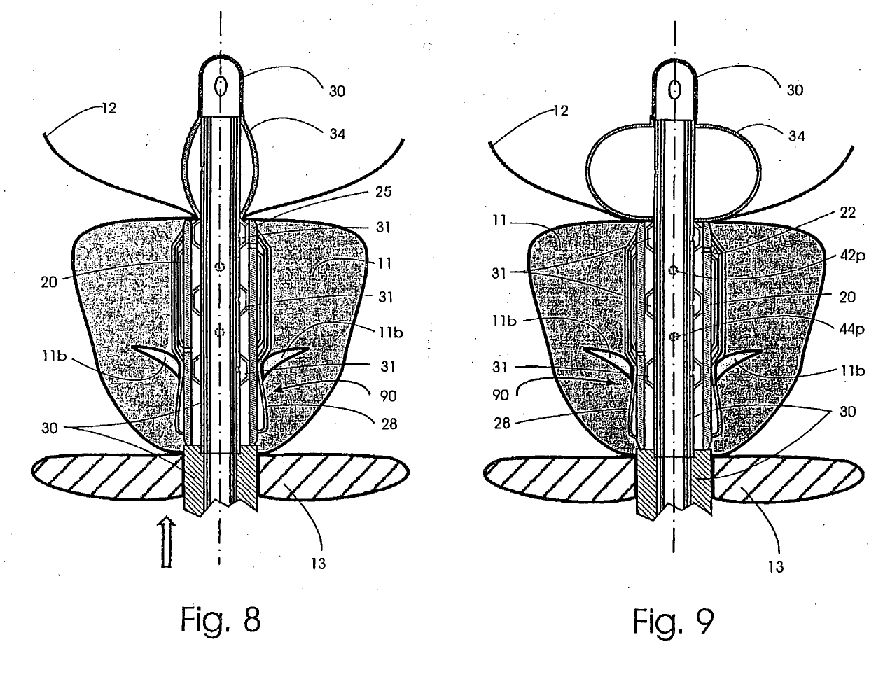

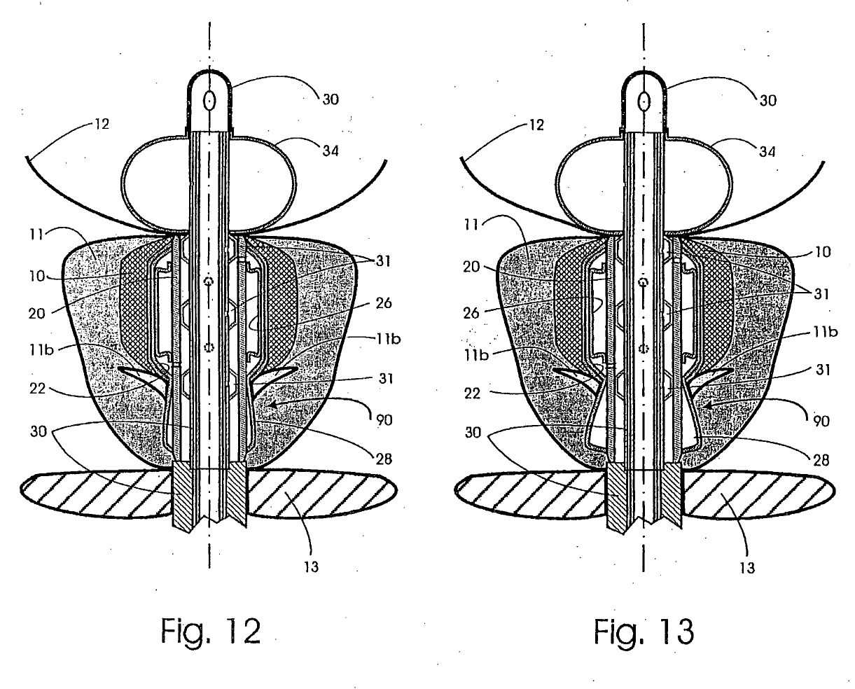

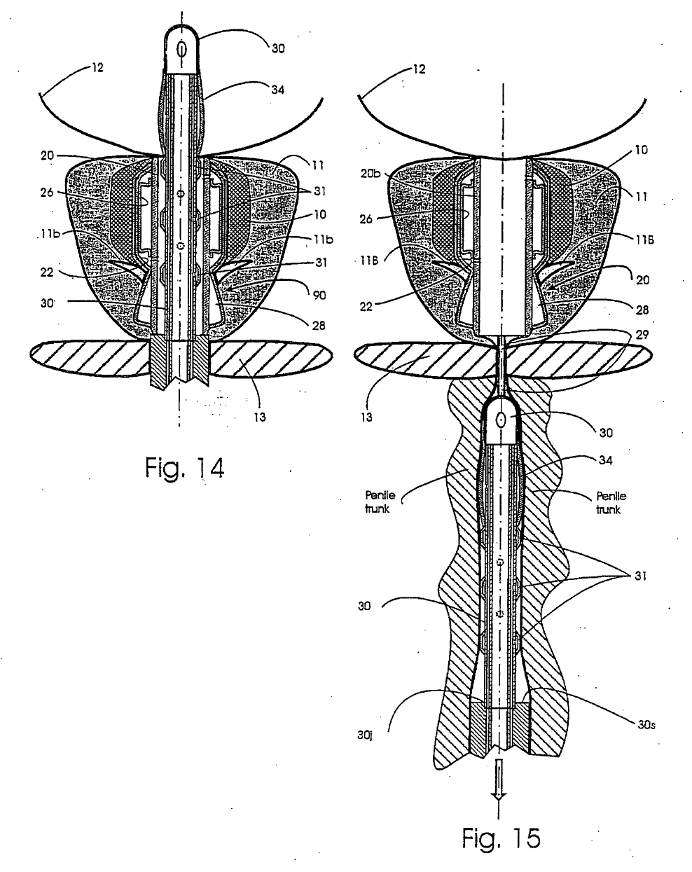

Turning now to FIG. 8, one embodiment of the present invention is shown. In this embodiment, the combination catheter 30 and stent 20 are afixed to each other, held by the fixation balloons 31. Together, they can be inserted into the prostate through the penile meatus (FIG. 8). Once the distal end of the combination device 90 enters the bladder of the subject, the distal anchoring balloon 34 is inflated (FIG. 9) to hold the device in position in the prostate relative to the bladder, with the treatment balloon 22 above the sphincter and, preferably, above the verumontanum 11b. FIG. 10 illustrates the initiation of the thermal therapy by continuously circulating fluid into and out of the treatment balloon 22 via a closed loop system thereby expanding same to contact adjacent tissue. The closed loop system may be a low volume fluid system (configured to circulate between 20 ml-100 ml) with an externally located pump and heating source or element. FIG. 11 illustrates the treatment balloon 22 collapsed after the thermal treatment has been completed and the circulating fluid interrupted or terminated. FIG. 12 illustrates the tissue-molding balloon 26 expanded to contact treated tissue and help inhibit closure of the prostatic urethra and FIG. 13 illustrates the localized tissue-anchoring balloon 28 expanded to help anchor the stent 20 in the subject. FIG. 14 illustrates the distal anchoring balloon 34 collapsed and the fixation balloons 31 also collapsed so that the treatment catheter member 30 is ready to be removed from the body. FIG. 15 illustrates the treatment catheter member 30 being slid away from the prostate leaving the stent member 20 in position.

In certain embodiments, as shown for example in FIG. 16, the catheter/stent 190 may have an open end and not employ a distal anchoring balloon. In addition, the stent member 20 may include other profile configurations, and is shown with only a treatment balloon 22 without a sleeve 24 or tissue-molding balloon 26. In other embodiments, the stent member 20 may be configured without expandable balloons.

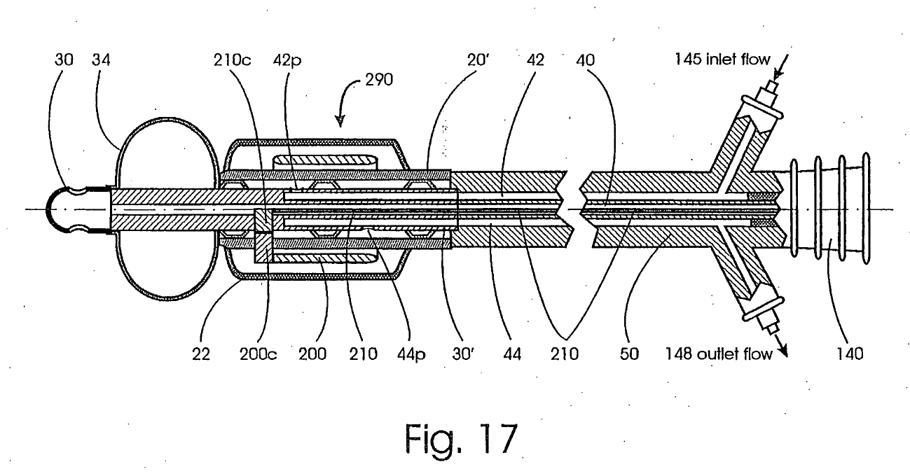

In some embodiments, as shown in FIG. 17, the catheter/stent assembly 290 includes a heating element 200, such as an electric heat source (i.e., a resistive heater, RF energy source, microwave energy source), a chemical energy heat source, thermal or optic or laser heating is located in the treatment balloon 22 (or on the stent member outer wall itself which may be used in some embodiments without a treating balloon). The electrical, thermal, optical, or chemical activation or generation connection line(s) can be carried in a lumen formed in the treatment catheter member 30′. The connection lines 210 can be configured to be in fluid isolation from the drainage channel 40 (not shown). The stent member 20′ includes a contact pad 200c which is operably associated with the heating element 200 and which extends inwardly a distance toward the treatment catheter member 30′. Similarly, the connection line 210 includes a contact pad 210c operably associated with the connection line and any outside activation or energy source. The connection line contact pad can extend outwardly toward the stent member 20′. In position, the contact pads 200c, 210c align and interconnect the connection line 200 to the heating element 210.

As shown in FIG. 18, the stent member 120 can be configured with a distal anchoring balloon 34 (instead of the treatment catheter member 30) and may also include a tissue anchoring balloon 28. In this embodiment, the stent member 120 provides a cavity 23, which can form part of the drainage channel 123 as the urine or fluid can enter the port 35 on the distal end portion thereof. Alternatively, the treatment catheter member 30 can be configured with an open end (not shown) which resides proximate to or below the distal anchoring balloon 34. Further, in certain embodiments, no tissue molding balloon 26 and/or localized tissue anchoring balloon 28 is required.

In addition, it is also noted that the use of an external sleeve 24 (FIG. 1A is also optional. Similarly, the use of fluid between the sleeve 24 and tissue molding balloon 26 (illustrated by the shading therebetween) as shown in FIGS. 7B and FIG. 10 is also optional.

As shown in FIG. 18, the stent member 120 may be configured with a closed end with at least one drainage and or fluid delivery (i e., flushing) orifices 35 above the anchoring balloon 34. In operation, the cavity 23 of the stent member 120 provides a central drainage lumen 123 which (when in operative position) can drain and/or flush fluids out of or into the lumen or body cavity.

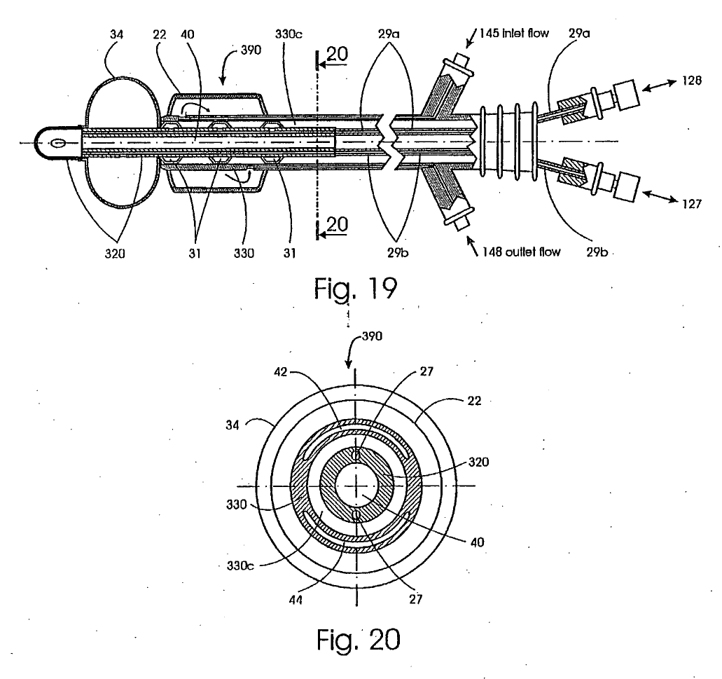

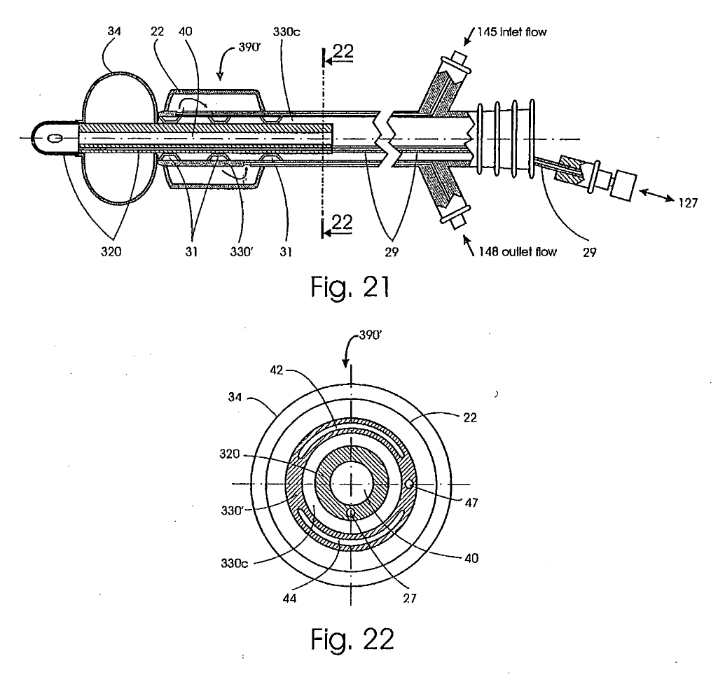

In certain embodiments, as shown in FIG. 19, the catheter/stent assembly 390 includes a stent member 320 that may be alternatively configured as the inner member while the treatment catheter member 330 is configured as the outer member which encases the underlying stent member 320. The inner stent member 320 resides inside the cavity of the outer treatment catheter member 330c. As shown in FIG. 19, the fixation balloons 31 are held on the stent member 320 and, when inflated, affix the stent member 320 to the outer treatment catheter member 330. Alternatively, in certain embodiments, as shown by the catheter/stent assembly 390′ in FIG. 21, the fixation balloons 31 may be positioned on the treatment catheter member 330′ so as to extend inwardly to attach the stent member 320. In addition, the stent member 320 can include the distal anchoring balloon 34 and provide the drainage and fluid delivery lumen 40. Certain embodiments of the treatment catheter member 330′ may include one or more fluid inlet passages. As shown in FIGS. 21 and 22, these may include an inlet lumen, an outlet lumen, and an inflation lumen 47.

In certain embodiments, the catheter/stent 90 (and the other assembly embodiments described herein 190, 290, 390, 390′) can be conformably configured such that it can follow the contours of the body cavity or lumen (such as the urethra) while having sufficient rigidity to maintain a sufficiently sized opening in the drainage lumen 40 to allow urine drainage and or flushing or drug delivery while in position.

In another embodiment, the stent body 20b is conformable but configured such that it is able to substantially maintain an opening in the central lumen when inserted and in position (and exposed to compressive swelling pressures in the localized treatment region) such that it maintains at least about 50-75% of the cross-sectional area, and preferably, at least about 75-90% or more of the cross-sectional area of the stent cavity 23 relative to its cross-sectional area prior to insertion in the urethra.

Of course, the cross-sectional shape of the lumen may alter from the non-inserted shape, depending on the pressure distribution of the tissue surrounding and contacting same. For example, during at least an initial healing period (after traumatic therapies such as surgical resection, the delivery of cyotoxic agents, and/or thermal ablation and the like) when in position in the prostate and exposed to prostatic tissue which is exhibiting distress, the stent body 20b is able to maintain a sufficient opening size to allow urine or body fluid drainage rates of above about 10 ml/min (preferably above about 20-25 ml/min.) when exposed to compressive pressures from the treated tissue on the order of about 7-21 psi. In certain embodiments, the stent body 20b is able to maintain this flow rate even after exposure to elevated temperatures above about 45° C. for at least about 10 minutes, and typically, for above about 30 minutes. Similarly, the stent body 20b can be configured to maintain the desired flow rate after cooling thermal treatments, including low temperature treatments (below about 10° C., 0° C., or even lower subzero or cryogenic temperatures). The stent members 20 of the instant invention can also be used to maintain an open passage of desired size for other treatments or applications where there is a desire to maintain the open passage in a flexible catheter which is exposed to edema or stress in the subject.

Examples of suitable materials for the stent member 20 and/or treatment catheter 30 are thermoplastic elastomers, silicone, rubber, plasticized PVC, or other suitable biomedically acceptable elastomeric body. In certain embodiments, the stent body is a unitary body 20b with a wall thickness of at least about 1.0 mm and a central lumen size of about 4.7-7.0 mm. As the prostate length can vary from subject to subject, the stent member 20 and/or the corresponding treatment catheter 30 may be produced in a plurality of lengths (such that the stent body has a range of from about 3-12 cm, and more preferably from about 4-10 cm).

The stent member 20 may include external indicia of axial or longitudinal movement which can alert the subject as to whether the stent 20 has migrated from its desired position. For example, a series of graduation marks can be attached to or formed on the external conduit (not shown). If the stent 20 moves toward the bladder 12, the subject can look at the applied graduation mark on the conduit 29 and recognize that it is migrating closer to the lumen entry point of the penile meatus; on the other hand, if the stent body 20b moves toward the sphincter 13, an increased number of markings will be visible and the conduit 29 with the applied mark will migrate away from the lumen entry.

The treatment catheter member 30 and/or stent member 20 can also be configured with radiopaque markers (not shown) to help identify its position for X-ray visualization. As such, X-rays can be taken at insertion/placement (initial positioning) and can also be taken periodically during the use of the stent or when there is a suspicion that the stent may have migrated from the desired location or merely to confirm proper positioning in the subject in situ. The radiopaque markers may be circumferentially arranged on the stent above and below the localized tissue-anchoring balloon 28 so that the anchoring balloon 28 can be more readily accentuated and confirmed to be in position in the X-ray (to affirm that it is located in the membranous urethra, above the sphincter as shown in FIG. 15). The radiopaque markers are applied to block the transmission of X-ray for better contrast in images. The opacity, degree of contrast, and sharpness of the image may vary with material and type of process used to create the marker. The radiopaque marker(s) may be arranged on the stent by any suitable biocompatible marker technique such as non-toxic radiopaque coatings, inks, thin-films, paints, tapes, strips, shrink tubing, and the like. See e.g., Richard Sahagian, Critical Insight: Marking Devices with Radiopaque Coatings, Medical Device & Diagnostic Industry (May, 1999), also available at URL www. devicelink.com/mddi/archive/99/05/011. Other examples of radiopaque markers include polyolefin inks available as No-Tox® Medical Device Polyolefin Inks from Colorcon located in West Point, Pa., and resin compounds with barium sulfate and/or bismuth such as is available from New England Urethane Inc. of North Haven, Conn. See also Danilychev et al., Improving Adhesion Characteristics of Wire Insulation Surfaces, Wire Technology International, March 1994 (discussing various treatments such as gas plasma treatment systems for medical products) which may be appropriate for use in the fabrication of the catheter or stent members 30, 20.

As the stent member 20 may reside in the body for long periods of times, typically between 2-21 (or 2-14) days (and potentially even longer, depending on the application), surface treatments or other treatments may also be applied to, or integrated into, the stent member 20 to achieve one or more of increased lubricity, low coefficient of friction (each for easier insertion) as well as increased tissue biocompatibility such as resistance to microbial growth and/or configured to reduce the incidence of UTI. In one embodiment, the stent body 20b comprises a material, at least on its externally exposed surfaces, which can inhibit the growth of undesirable microbial organisms while the stent member 20 is held in the body during (and after) the healing period as described herein.

The stent member 20 may also or alternatively be coated with a biocompatible antimicrobial solution or coating which can inhibit the growth of bacteria, yeast, mold, and fungus. One suitable material may be the antimicrobial silver zeolite based product available from HealthShield Technologies LLC of Wakefield, Mass. Another alternative is a Photolink® Infection Resistance antimicrobial coating or a hemocompatible coating from SurModics, Inc. of Eden Prairie, Minn. The coating may also include other bioactive ingredients (with or without the antimicrobial coating), such as antibiotics, and the like. One product is identified as LubriLAST™ lubricious coatings from AST of Billerica, Mass.

Additionally, or alternatively, the stent member 20 (and/or treatment catheter member 30) can be configured with a biocompatible lubricant or low-friction material to help reduce any discomfort associated with the insertion of the device into the body. Coatings which may be appropriate include coatings which promote lubricity and wetability. For example, a hydrophilic coating which is applied as a thin (on the order of about 0.5-50 microns thick) layer which is chemically bonded with UV light over the external surface of the member 20, 30. One such product is a hydrophilic polymer identified as Hydrolene® available from SurModics, Inc., of Eden Prairie, Minn. Other similar products are also available from the same source. Still further, the stent member 20 can be configured not only to provide the lubricious coating but to also include bioactive ingredients configured to provide sustained release of antibiotics, antimicrobial, and anti-restenosis agents, identified as LubrilLast™ from AST as described above.

In certain embodiments, the thermal treatment can be carried out by exposing the targeted tissue in the body to thermocoagulation introduced by the combination catheter/stent 90 when configured to direct circulating hot liquid (which may be heated external of the body of the subject) to the targeted treatment region. For thermal ablation procedures, the tissue is exposed to an elevated temperature that is greater than or equal to about 45° C. for a predetermined period of time. In certain embodiments, the thermal ablation is directed to treating BPH. It is also preferred that the prostatic tissue is exposed to a temperature which is at about 60-62° C. for a treatment period which is about 40-60 minutes, and typically about 45 minutes.

Referring now to FIG. 15, the thermal ablation treatment region 10 is indicated by the crosshatched region in the prostate 11. The term “thermal ablation” refers to exposing the targeted tissue to a temperature that is sufficient to kill the tissue. As shown in FIG. 15, high temperature thermal ablation therapy is carried out by causing the localized targeted tissue to thermocoagulate via contact with the expandable treatment balloon 22 residing on the stent member 20 cooperating with an underlying treatment catheter member 30 which are held together and inserted into the subject to direct circulating hot liquid heated external of the body of the subject to the targeted treatment region within the biological subject. As shown, the targeted tissue is the prostatic urethra, the treatment region 10 being generally described as including the upper portion of the urethra in the prostate (the prostatic urethra) so as to extend generally below the bladder neck and above the verumontanum 11b of the subject. Alternatively, the treatment region 10 may include the bladder neck or a portion of the bladder neck itself.

A suitable thermal treatment system employing water-induced thermotherapy for prostate treatments is identified as the Thermoflex® System available from ArgoMed, Inc. of Cary, N.C. The combination catheter/stent can be configured to operate with circulating fluid heated external of the body and circulated in the combination catheter/stent similar to conventional treatment catheters also available from ArgoMed, Inc. See also, U.S. Pat. Nos. 5,257,977 and 5,549,559 to Eshel, and co-pending, co-assigned U.S. patent application Ser. No. 09/433,952 to Eshel et al., the contents of which are hereby incorporated by reference as if recited in full herein. Alternatively, as noted above, the catheter/stent can be configured to operate with alternative thermal generation means (whether local or remote) and whether heat or cooling or both, such as RF, microwave, laser, refrigeration means, and the like.

As shown by the arrows in FIGS. 3, 7A, 10, 19, and 21, the circulating fluid is directed into the treatment balloon 22 that then expands in response to the quantity of fluid held therein. Preferably, a low volume (meaning below about 100 ml, preferably below about 50 ml, and more preferably below about 20 ml) of circulating heated fluid is circulated, during operation, at any one time, through a closed loop system to deliver the thermal ablation treatment via the treatment catheter/stent 90. The circulating fluid (and the balloon inflation media) is preferably selected to be non-toxic and to reduce any potential noxious effect to the subject should a situation arise where the balloon integrity may be compromised, accidentally rupture, leak, or otherwise become impaired during service.

Various prophylactic antibiotics can also be delivered systemically such as orally, before and/or after a thermal treatment or thermal ablation session. In other embodiments, antibiotics or anti-inflammatory (including non-steroidal and α-blockers, Cox-inhibitors, or antioxidants) or other selected drugs, can be delivered directly into the treatment region (such as via the drainage channel 40, permeable treatment balloons 22, or medication port 92 (FIG. 16). This can result in reduced catheterization time and reduced incidence of urinary tract infections (UTI). Antibiotics known as RIFAMPIN, MINOCYCLINE, and VANCOMYCIN or others have been successfully used in certain medical or clinical sites. CELEBREX has also been used in conjunction with WIT of the prostatic urethra (given before and/or after the thermal ablation treatment). Nitrofuratoin (trade name MACRODANTIN) has been incorporated into the catheter itself to treat UTI and to promote faster healing. Alpha-blockers such as FLOMA, CARDURA, and HYTRIN have also been used, as well as other agents such as DETROL, DITROPAN XL, and PYRIDIUM.

Examples of other anti-inflammatory medicines which may be used either locally and/or systemically with thermal treatments and thermal ablation therapies include, but are not limited to, steroids, nonsteroidal anti-inflammatory drugs such as tolmetin (trade name TOLECTIN), meclofenamate (trade name LEFLUNOMIDE), meclofenamate (trade name MECLOMEN), mefenamic acid (trade name PONSTEL), diclofenac (trade name VOLTAREN), diclofenac potassium (trade name CATAFLAM), nabumetone (trade name RELAFEN), diflunisal (trade name DOLOBID), fenoprofen (trade name NALFON), etodolac (trade name LODINE), ketorolac (trade name TORADOL) and other anti-inflammatory drugs such as leflunomide, rofecoxib (trade name VIOXX), ibuprofin (such as MOTRIN) and celecoxib (trade name CELEBREX). Other types of medicines or drugs can also be used such as anti-hypertensive drugs including terazosin (trade name HYTRIN), doxazosin (trade name CARDURA), and immunosuppressive drugs including cyclosporine (trade name SANDIMMUNE or NEORAL).

Examples of antibiotics which may be suitable for use in conjunction with thermal treatments including thermal ablations, include, but are not limited to, cipro, levaquin, septra, gentamycin, clindamycin (trade name CLEOCIN), azithromycin (trade name ZITHROMAX), trimethoprimn (trade name TRIMPEX or PROLOPRIM) norfioxacin (trade name NOROXIN).

In each of the embodiments described herein, the catheter and coatings are preferably configured to withstand suitable sterilization processes as they will be used in medical applications.

In certain embodiments of the present invention, at a desired time, typically proximate to removal of the stent member 20, biodegradable materials may be administered as flowable fluids through the drainage channel 40 or medicament port 92 (FIG. 16) onto the treated tissue or targeted region. These flowable fluids or solutions, when subjected to different conditions, harden or solidify to form a localized shell which can provide a biodegradable stent for the treated region. For example, polymerizing gels that solidify upon contact with body fluids can be inserted into the subject to the treated region. In operation, these gels can flow about the catheter body and form in situ, a protective shell or coating about the targeted region. The biodegradable materials can also be a combination of two polymers that solidify when they come into contact with each other. In this way, a biodegradable stent may be used to complement or potentially enhance the therapy efficacy to provide adequate urinary passage openings for increased post-treatment periods.

Typical bio-absorbable materials used in urology include high molecular weight polymers of polylactic or polyglycolic acid. Some of these materials are thought to have been used in Finland after laser ablation treatment of the prostate as well as after trans-urethral microwave therapy, and for recurrent bulbous urethral strictures. See Isoltalo et al., Biocompatibility testing of a new bioabsorbable X-ray positive SR-PLA 96/4 urethral stent, Jnl. Of Urol., pp. 1764-1767, Vol. 162 (1999). Some of the bio-absorbable materials or gels may also be used as drug delivery systems with (typically after) thermocoagulation treatments. Examples materials which may be suitable to act as biodegradable or bioabsorbable stents include: hydrogel polymers which solidify at high temperatures after they are injected in liquid form; THOREX, an albumin base polymer which can purportedly adhere to tissue in less than about 15 seconds; recombinant collagens, human proteins which provide may reduce immune reactions or transfer of pathogens from animal-based materials; BST-GEL which is in a liquid state at low temperature and at a solid state at body temperature and which may be used for drug release; COSEAL, a synthetic self-polymerizing gel, which is a mixture of collagen and polyethylene glycol allegedly resorbable within 30 days and capable of drug delivery; and ATRIGEL, a biodegradable polymer system which can be applied to tissue as a liquid which then solidifies upon contact with the body's moist environment and which has the ability to time-release different drugs. For additional description of suitable biodegradeable stents, see co-pending and co-assigned U.S. patent application Ser. No. 10/011,494, identified by Attorney Docket No. 9149-20, incorporated by reference hereinabove.

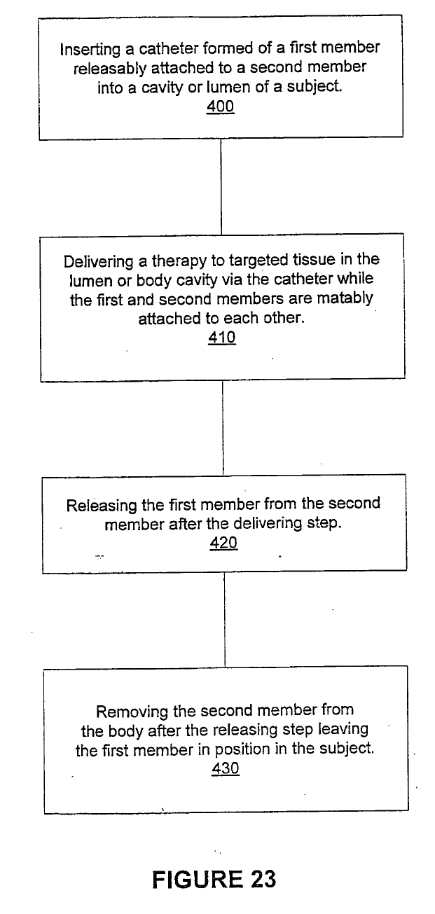

Turning now to FIG. 23, a block diagram illustrates certain embodiments of a method of administering a therapy to a natural lumen or body cavity of a subject. As shown, the method includes: (a) inserting a catheter formed of a first member releasably matably attached to a second member into a body cavity or lumen of a subject (Block 400); (b) delivering a therapy to targeted tissue in the lumen or body cavity via the catheter while the first and second members are attached to each other (Block 410); (c) releasing the first member from the second member after said delivering step (Block 420); and (d) removing the second member from the body of the subject such that the first member remains in position in the subject (Block 430). In some embodiments, the therapy is delivered by a cooperating fluid transfer relationship between the first and second members.

The therapy can include one or more of administration of a drug or thermal treatment or other desired therapy. The first member may include drug delivery ports, surface recesses or channels, lumens and/or permeable expandable balloons (membranes) to help disperse the desired drug.

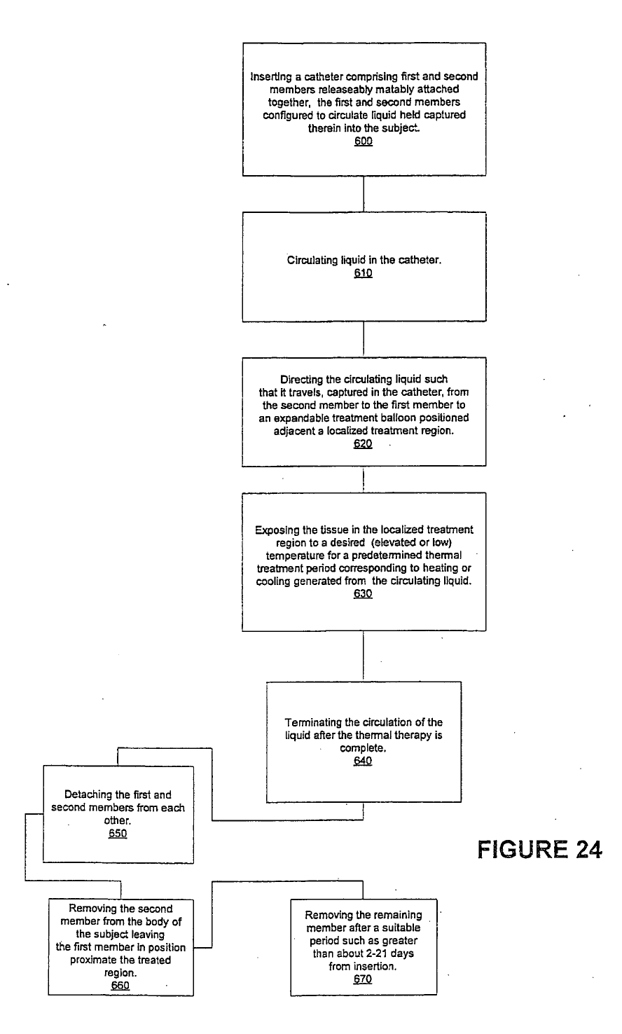

FIG. 24 is a block diagram of a method for treating a region in the natural lumen or body cavity of a subject. The method includes inserting a catheter comprising first and second members releasably matably attached together so as to cooperate to circulate the liquid therebetween, the first and second members when attached being configured to circulate captured liquid to desired region of the subject (Block 600) and circulating liquid in the catheter (Block 610). In position, the stent wall and catheter wall can be'sized to be spaced-apart and held together by radially expandable fixation balloons. For thermal ablation applications, the circulating heated liquid is heated to a temperature above about 45° C. (some embodiments may heat the liquid external of the body and pump it through a closed loop system). Other thermal applications may heat or cool the liquid (or both) over the course of the treatment session.

The circulating heated liquid is directed through the catheter such that it travels, captured in the catheter, from the second member to the first member to a treatment balloon, positioned adjacent a localized treatment region in the body of the subject (Block 620). The tissue in the localized treatment region is exposed to a desired temperature for a predetermined thermal treatment period corresponding to exposure to the cooling or heating generated from the circulating liquid (Block 630). (The method may optionally include the step of insulating the non-targeted tissue below the targeted tissue in the treatment region such that the non-targeted tissue positioned there is exposed to a maximum temperature of about 42° C. (or from cooler temperatures during cooling therapies) from contact with the treatment catheter during the exposing step). In any event, after the thermal therapy is completed, circulation of the liquid can be terminated (Block 640). The first and second members of the catheter can be detached from each other (Block 650). The second member is removed from the body, leaving the first member in position proximate the treated region (Block 660). The first member may be removed at a suitable later time, such as after a time greater than about 2-21 (typically within 2-21) days from insertion (Block 670).

In certain embodiments, the method may be used to treat BPH or prostatitis, or other urinary or body conditions. In addition, for embodiments employing tissue-molding balloons, the pressure can be adjusted during the time the stent member is in the body (to increase or decrease as needed to adjust the flow rate or opening size of the lumen) to facilitate shaping or molding the treated tissue as the tissue reacts to the treatment (swelling, edema, etc). The stent can be biodegradable or non-biodegradable. The stent member as well as the catheter can include medications and other surface treatments as discussed above.

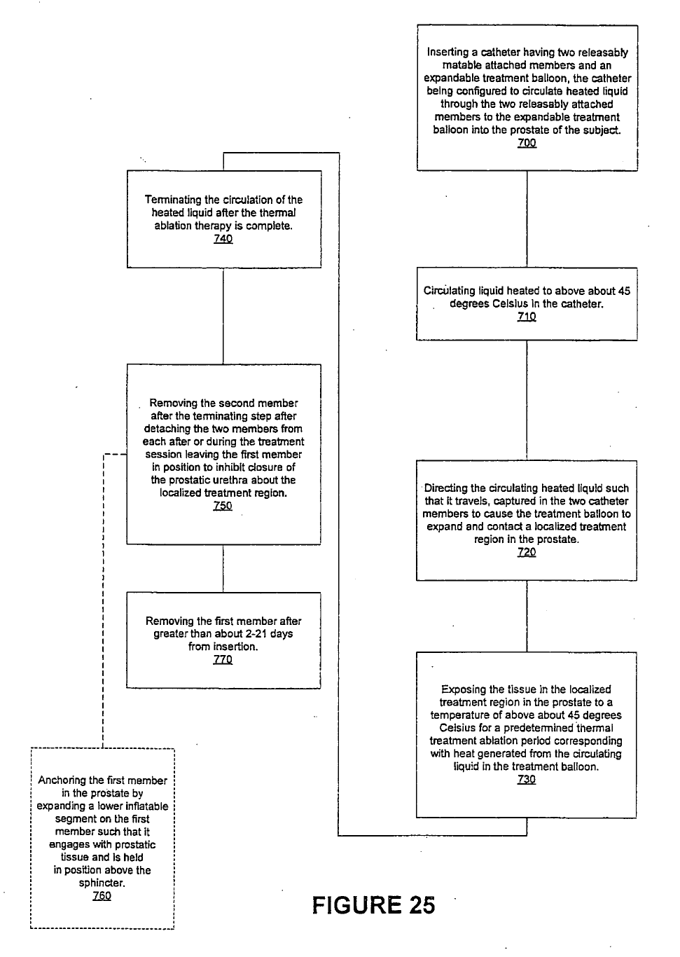

FIG. 25 is a block diagram that is similar to the method described in FIG. 24, but is particularly directed to prostatic applications, and may be used for treating BPH according to the present invention. The method includes inserting a catheter having two releasably attached members and an expandable treatment balloon, the catheter being configured to circulate heated liquid through the two realeasably attached members to the expandable treatment balloon into the prostate of the subject (Block 700) and then circulating liquid heated to above 45° C. in the catheter (Block 710). The circulating heated liquid is directed such that it travels captured in the two catheter members to cause the treatment balloon to expand to contact a localized treatment region in the prostate (which may circulate the liquid as it is held captured in the catheter, through the penile meatus, along the penile urethra, the bulbous urethra, and the membranous urethra) (Block 720). The tissue in the localized treatment region in the prostate is exposed to a temperature above about 45° C. for a predetermined thermal ablation treatment period by exposure to the heated circulating liquid (typically at about 60° C. for more than about 30 minutes) (Block 730). As noted above, preferably, the localized treatment region is the prostatic urethra, leaving the membranous urethra, non-ablated. This may be accomplished in circulating systems (which heat remotely) by insulating the shaft of the treatment catheter up to the treatment balloon to inhibit the exposure of non-targeted tissue to ablation temperatures. The circulation of the heated liquid is terminated after thermal ablation therapy is complete (Block 740) and the second member is removed after the terminating step after detaching the two members from each other during or after the treatment session, leaving the first member in position to inhibit closure of the passage of the prostatic urethra about the treatment region (Block 750). The first member can be removed after greater than about 2-14 (typically within 2-21) days from insertion of the catheter (Block 770). Optionally, the first member can be anchored in the prostate by expanding a localized anchoring balloon to hold the first member above the sphincter (Block 760).

It will be understood that certain of the blocks of the block diagrams and combinations of blocks in block diagram figures can be implemented or directed to be carried out by computer program instructions. These computer program instructions may be loaded onto a computer or other programmable data processing apparatus to produce a machine, such that the instructions which execute on the computer or other programmable data processing apparatus create means for implementing the functions specified in the flowchart block or blocks. These computer program instructions may also be stored in a computer-readable memory that can direct a computer or other programmable data processing apparatus or associated hardware equipment to function in a particular manner diagrams.

Although described herein primarily for use as a prostatic thermal treatment catheter, it will be appreciated by those of skill in the art that the instant invention may be configured in catheter configurations adapted for non-thermal as well as non-circulating uses, and for other natural lumens or body cavities such as blood vessels (including, but not limited to, arteries) the rectum, the colon, the female reproductive system, such as the cervix, uterus or fallopian tubes, the throat, the ear, the nose, passages of the heart and/or associated valves, the respiratory system, the digestive system, and the like.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A combination treatment catheter and post-treatment stent device configured for insertion into a body cavity and/or lumen of a biological subject, the device comprising:
a first member having an inner cavity; and
a second member, at least a portion of which is sized and configured to be received within said first member cavity so as to be in cooperating alignment with and releaseably secured to said first member,
wherein, when secured together, said first and second members cooperate to define a treatment catheter that administers a selected in vivo treatment to the subject at a desired operative treatment location, and wherein said first and second members are configured to release from each other in vivo so that a selected one of said first and second members is independently removeable from the subject while the other of said first and second members is adapted to remain in the subject as a post-treatment stent held proximate the operative treatment location for a desired post-treatment duration.

2. A device according to claim 1, wherein said first and second members each have a wall with a respective outer surface and an opposing inner surface, wherein, when secured together, said first and second members define at least one enclosed chamber extending laterally between said second member wall and said first member wall, the at least one enclosed chamber defining a fluid passage which, in operation, allows a selected exogenously introduced fluid to flow between said first and second members.

3. A device according to claim 2, wherein said second member further comprises a plurality of fixation balloons positioned to extend outwardly from said outer surface of second member wall to releasably engage with the inner surface of said first member when said second member is aligned with and in position in said first member cavity to define a plurality of enclosed fluid passages extending between said first and second member walls.

4. A device according to claim 3, wherein said first member further comprises:
at least one inflatable treatment balloon positioned to expand outwardly away from the outer surface of said first member wall, such that when inflated, in operative position and configuration, said treatment balloon expands outwardly to contact targeted tissue of the subject at the operative treatment location; and
at least one inlet port extending through the wall of said first member and being in fluid communication with at least one of the enclosed fluid passages and the at least one treatment balloon,
wherein, in operation, when said first and second members are attached together to define the treatment catheter, the at least one inflatable-treatment balloon expands responsive to the flow of the exogeneously introduced fluid into the at least one inlet port.

5. A device according to claim 4, wherein said second member wall is an axially extending wall having at least one inner fluid lumen encased therein that extends along a length thereof, wherein said second member further comprises:
a plurality of inflation ports, at least one inflation port extending between an inflation path in the second member and a respective one of the fixation balloons; and
an outlet port in fluid communication with the at least one inner fluid lumen in the second member wall and extending through said second member wall.

6. A device according to claim 5, wherein, in operation, the exogenously introduced fluid flows from a location that is external of the subject to, in serial order, said second member inner fluid lumen, through said second member outlet port, into the at least one enclosed chamber, into said inlet port of said first member and to said at least one treatment balloon to expand said at least one treatment balloon responsive to the introduction of the fluid therein.

7. A device according to claim 1, wherein said second member is configured as an elongated tubular body with an upper portion and a lower portion, the lower portion having an increased cross-sectional width relative to the upper portion, the lower portion including a thermally insulated region extending along a length thereof.

8. A device according to claim 3, wherein each of the plurality of fixation balloons expands to securely and separately contact and seal against the inner surface of the wall of the first member.

9. A device according to claim 6, wherein the second member at least one inner fluid lumen is a plurality of inner fluid lumens, at least one of which is a drainage or fluid delivery lumen.

10. A device according to claim 9, wherein said second member plurality of inner fluid lumens includes at least one circulating fluid inlet lumen and at least one circulating fluid outlet lumen.

11. A device according to claim 10, wherein the first member further comprises an outlet port extending through the wall thereof positioned such that the first member outlet port is spatially separate from the first member inlet port and is in fluid communication with the inflatable treatment balloon, and wherein the second member further comprises a return port in fluid communication with the circulating fluid outlet lumen and extending through the second member outer wall.

12. A device according to claim 11, wherein said second member has an elongated tubular body, wherein said second member plurality of inner fluid lumens all axially extend within said second member tubular body such that the plurality of inner fluid lumens are encased by said wall of said second member and, for at least a portion of the length of said second member tubular body, the plurality of inner fluid lumens are held inside said second member such that they are separated from the outer surface of said second member wall by an increased thermal insulation region.

13. A device according to claim 11, wherein, when said first and second members are aligned and engaged, each of said plurality of fixation balloons expands outwardly away from said second member to securely contact said first member to provide a plurality of separate enclosed chambers extending between said second member wall and said first member wall so as to define at least two discrete fluid passages in fluid isolation from each other, one of which allows fluid to enter into said treatment balloon by traveling from said second member circulating inlet fluid lumen to said second member outlet port through a selected one of the enclosed chambers to said first member inlet port and into said treatment balloon, and the other of which allows fluid to exit said treatment balloon through said first member outlet port to a different one of the enclosed chambers into said second member return port and into said second member-circulating fluid outlet lumen.

14. A device according to claim 6, wherein the second member further comprises an inflatable anchoring balloon positioned a distance above the plurality of fixation balloons, wherein the inflatable anchoring balloon expands outwardly a width which is greater than the cross-sectional width of the wall of said first member, and wherein said second member includes at least one inflation path in fluid communication with the inflatable anchoring balloon and, during operation, with an externally located inflation source.

15. A device according to claim 1, wherein said first and second members have associated lengths, the length of said first member being at least about 50% less than that of said second member, wherein said first member is configured to be the stent member, and wherein said first member farther comprises a tissue engaging, inflatable, localized anchoring balloon located so as to be able to outwardly expand to contact tissue when in operative position in the subject.

16. A device according to claim 6, wherein said first member has a contouring sleeve disposed over the at least one inflatable treatment balloon such that each is concurrently responsive to the introduction of fluid into the treatment balloon.

17. A device according to claim 16, wherein a quantity of the exogeneously introduced fluid is captured in said first member between the at least one treatment balloon and the contouring sleeve to enhance thermal transmissivity therebetween.

18. A device according to claim 1, wherein said first member has a length, an outer wall, and opposing inner and outer surfaces and is the stent member that is adapted to remain in the body upon removal of said second member, and wherein said first member further comprises:
at least one inflatable treatment balloon positioned to expand outwardly away from said first member outer wall, such that, in operative position and configuration, when inflated, said at least one treatment balloon expands outwardly to contact targeted tissue of the subject at the operative treatment location, wherein, in operation, when said first and second members are attached together to define the treatment catheter, the at least one inflatable treatment balloon expands responsive to the flow of the exogeneously introduced fluid;
a tissue-molding balloon disposed between said first member outer wall and the inflatable treatment balloon and configured to expand outwardly away from said first member outer wall; and
a tissue molding balloon inflation path directed along at least a portion of the length of said first member outer wall, the tissue-molding balloon path being in fluid communication with an externally located inflation source,
wherein, in operation, the tissue-molding balloon is separately expandable from the at least one inflatable treatment balloon, and wherein, in operative position in the body, the tissue-molding balloon is configured to expand to contact the at least one inflatable treatment balloon and cause the at least one treatment balloon to contact adjacent tissue.

19. A device according to claim 18, wherein said first member further comprises:
a localized tissue anchoring balloon configured to outwardly expand away from the outer wall of said first member; and
at least one axially extending conduit having a cross-sectional width which is substantially smaller than the cross-sectional width of the first member and a length which is sufficient to be accessible external of the subject's body, wherein the at least one conduit is in fluid communication with the localized tissue anchoring balloon, and wherein the at least one conduit is affixed to said first member with an attachment strength sufficient to allow said first member to be extricated from the body of the subject by pulling on the conduit to dislodge and remove said first member therefrom.

20. A device according to claim 19, wherein said first member is configured to reside in the prostatic urethra of the subject above the urethral sphincter such that, in position, the at least one conduit extends through the urethral sphincter so that the sphincter is able to function normally.

21. A device according to claim 3, wherein the exogenously introduced fluid is a thermally treated liquid.

22. A device according to claim 3, wherein the exogenously introduced fluid comprises a therapeutic liquid.

23. A device according to claim 1, wherein said first member has axially spaced apart upper and lower portions, wherein said second member has an elongated tubular body with at least one fluid lumen therein and an upper portion that is configured to reside substantially within the cavity of said first member and oriented so that said first and second member upper portions are proximate each other, and wherein said second member tubular body has an intermediate portion that is axially spaced apart from and resides below said second member upper portion, said second member intermediate portion is configured to align with and seal against said first member lower portion so as to be able to sealably direct a thermally treated liquid to travel from the fluid lumen of said second member through an enclosed chamber defined by said first and second members and into the treatment balloon of said first member.

24. A device according to claim 3, wherein said second member comprises a distal anchoring balloon positioned above the plurality of fixation balloons.

25. A device according to claim 1, wherein said first member further comprises at least one axially extending conduit having a cross-sectional width which is substantially smaller than the cross-sectional width of said first member and a length which is sufficient to be accessible external of the body, and wherein said second member includes an outer wall with at least one recess configured and sized to releasably hold the at least one conduit therein such that the conduit is inhibited from movement while the second and first members are matably secured together and so that the conduit is able to automatically exit the recess upon removal of said second member from the subject's body to allow said first member and conduit to remain in position in the subject's body after said second member is removed.

26. A device according to claim 1, wherein the second member comprises a fluid drainage and delivery lumen, wherein the device is configured such that it is sufficiently conformable to yield to the contours of the subject's body as it is inserted therein with said first and second members attached together, yet sufficiently rigid to maintain the drainage and delivery lumen in an open condition sufficient to allow fluid flow therethrough when in position in the subject's body and exposed to pressure from tissue which is exhibiting distress during or after a therapy.

27. A combination treatment catheter/stent device configured for insertion into a body cavity or lumen of a biological subject, the treatment catheter/stent comprising:
a flexible elongated tubular body having a first member and a releasably attached second member, wherein, when attached and in position in a subject, said first and second members cooperate to generate and deliver a desired therapy to tissue in a localized region of the body of the subject, and wherein after the desired therapy has been delivered, the first and second members are disengagable from each other while they are held in vivo and a selected one of the members is slidably removable from the body of the subject while the other member remains in position proximate the treated tissue.

28. A device according to claim 27, wherein said first member is a female member with an inner wall and said second member is a male member with an outer wall configured to be received into the female member, the female member comprising an inflatable treatment balloon positioned about a peripheral distal portion such that the treatment balloon is expandable to a configuration which extends radially outward a distance from the outer wall of the female member.

29. A device according to claim 28, wherein the male member includes a plurality of lumens axially extending within the male member, and wherein the male member includes an outer wall and a region having increased thermal insulation such that the plurality of lumens are encased by the outer wall and, for at least a portion of the length of the fluid lumens, by the increased thermal insulation region.

30. A device according to claim 29, wherein the plurality of lumens include a circulating fluid inlet lumen, a circulating fluid outlet lumen, and a drainage and fluid delivery lumen, and wherein, when the male and female members are matably attached, the fluid inlet and circulating fluid outlet lumens are in fluid communication with the treatment balloon and are configured, in operation, to cause the treatment balloon to expand.

31. A device according to claim 30, wherein the male member includes a plurality of fixation balloons configured to expand to contact the female member to hold the female and male members securely together, wherein, when secured together, the fixation balloons, the inner wall of the female member, and the outer wall of the male member define a plurality of liquid flow path channels in fluid isolation from each other.

32. A device according to claim 31, wherein, in position in the subject, the device is configured to circulate a liquid heated external of the subject's body and direct it to travel, held captured in the device, through the inlet lumen in the male member to a first of the enclosed chambers to the treatment balloon and out through a different one of the enclosed chambers to the male member outlet lumen, the liquid being heated to a temperature sufficient to thermally treat and/or ablate tissue at a desired biological target site proximate the treatment balloon.

33. A device according to claim 32, wherein, in operation, an outer surface of the outer wall of the male member overlying the increased thermal insulation region has a temperature which is below 45° C.

34. A device according to claim 33, wherein the temperature of the heated circulating liquid as it enters tie male member inlet lumen is about 60° C., and wherein, measured ex vivo, the outer surface of the outer wall about the increased thermal insulation region exhibits a maximum temperature of about 42° C. after a thermal treatment period of at least 30 minutes.

35. A device according to claim 34, wherein the drainage and delivery lumen is centrally disposed within the male member, and wherein the fluid inlet and circulating fluid outlet lumens are positioned on opposing sides thereof.

36. A device according to claim 29, wherein the plurality of fluid lumens comprises at least one drainage and fluid delivery lumen, and wherein the combination catheter/stent tubular body is sufficiently conformable to yield to the contours of the subject's body as it is inserted therein, yet sufficiently rigid to maintain the drainage and delivery lumen in an open condition which is sized at about at least 50-75% of the size of the lumen outside the subject's body before the treatment when in position in the subject's body and exposed to tissue which is exhibiting distress during or subsequent to delivery of a predetermined treatment.

37. A device according to claim 29, wherein the plurality of fluid lumens comprises at least one drainage and fluid delivery lumen, and wherein the device is sized and configured for insertion into the urethra of a male subject, the urethra generally including, in serial order from the external most portion to the internal portion, the penile meatus, the penile urethra, the bulbous urethra, the sphincter, the membranous urethra, the prostatic urethra, the bladder neck and the bladder, wherein the device is sufficiently conformable to yield to the contours of the subject's body as it is inserted therein, yet sufficiently rigid to maintain said drainage and delivery lumen in an open condition sufficient to discharge urine at a flow rate of at least about 20 ml/min when the device with the two members attached together and/or the male member alone, is in position in the urethra and exposed to prostatic tissue which is exhibiting distress during or subsequent to undergoing thermal ablation therapy.

38. A device according to claim 27, wherein said first member is a female member with an inner wall and said second member is a male member with an outer wall configured to be received into the female member, wherein the female member further comprises at least one axially extending conduit attached thereto, wherein, in position, the female member resides above the sphincter and the conduit extends therethrough so that the sphincter can operate in a substantially normal manner while the female member resides in the body of the subject, and wherein the at least one conduit is affixed to the female member with an attachment strength sufficient to allow the female member to be extricated from the body of the subject by pulling on the conduit to dislodge and remove the female member therefrom.

39. A device according to claim 27, wherein said first member is a female member with an inner wall and said second member is a male member with an outer wall configured to be received within the female member, wherein the male member further comprises at least one axially extending conduit attached thereto, wherein, in position, the male member resides above the sphincter and the conduit extends therethrough so that the sphincter can operate in a substantially normal manner while the male member resides in the body of the subject, and wherein the at least one conduit is affixed to the male member with an attachment strength sufficient to allow the male member to be extricated from the body of the subject by pulling on the conduit to dislodge and remove the male member therefrom.

40. A device according to claim 27, wherein said first member is a female member with an outer wall and said second member is a male member with an outer wall configured to be received into the female member, wherein the female member comprises an inflatable treatment balloon configured to expand outwardly from the female member outer wall, wherein the male member comprises an anchoring balloon positioned on a distal portion thereof such that it is more distal than the treatment balloon when the female and male members are secured together, the anchoring balloon configured and sized such that, when inflated and in position in the biological subject, the anchoring balloon resides against the bladder neck of the subject to position the treatment balloon on the female member in the prostate relative to the bladder of the subject.

41. A device according to claim 40, wherein the male member includes a port on said distal portion thereof in fluid communication with a drainage and delivery lumen to allow urine to drain therethrough.

42. A method of thermally treating a subject, comprising:
inserting a flexible combination treatment catheter/stent into a natural lumen or body cavity of a subject, the combination catheter/stent comprising an elongated male member and an elongated female member, wherein the male member includes at least one inflatable fixation balloon configured to expand to contact and securely attach the female and male members together such that they are operatively associated during delivery of a desired thermal treatment to the lumen or body cavity;
delivering a desired thermal treatment to a target site in the lumen or body cavity from the combination treatment catheter/stent;
collapsing the at least one fixation balloon to disengage the male member from the female member after said delivering step;
removing the male member from the body of the subject after the delivering and collapsing steps, wherein the female member is maintained in position in the subject during the collapsing and removing steps.

43. A method according to claim 42, wherein the female member has a first length and the male member has a second length, the second length being at least about 50% longer than the first length.

44. A method according to claim 42, wherein the female member comprises an inflatable treatment balloon thereon, and wherein an outer wall of the male member, an inner wall of the female member and the at least one fixation balloon define at least one enclosed fluid passage chamber.

45. A method according to claim 44, wherein the delivering step comprises circulating thermally treated liquid, heated external of the subject to above about 45° C., in the combination treatment catheter/stent such that the heated liquid serially travels, captured in the male member, into at least one of the enclosed chambers, then into the inflatable treatment balloon on the female member, to thereby expose targeted tissue to a temperature of above about 45° C. for a predetermined thermal ablation treatment period.

46. A method according to claim 44, wherein the delivering step comprises circulating liquid, cooled external of the subject to below about 10° C., in the combination treatment catheter/stent such that the cooled liquid serially travels. captured in the male member, into at least one of the enclosed chambers, then into the inflatable treatment balloon on the female member, to thereby expose targeted tissue to a temperature of below about 10° C. for a predetermined thermal treatment period.

47. A method according to claim 45, further comprising insulating non-targeted tissue below the targeted region such that the non-targeted tissue is exposed to a maximum temperature of about 42° C. from contact with the external surface of the male member of the combination treatment catheter/stent during the delivering step.

48. A method according to claim 45, further comprising draining body fluids through the combination treatment catheter/stent during the delivering step.

49. A method according to claim 42, wherein the removing step is carried out after the delivering step during a treatment session.

50. A method according to claim 42, further comprising extricating the female member after a desired healing period of between about 2-21 days after the delivering step.

51. A method according to claim 42, wherein the female member comprises at least one axially extending conduit attached thereto, the conduit having length sufficient to extend out of the body when the female member is in position therein, the method further comprising extricating the female member by pulling on the conduit to dislodge and extricate the female member from the body after the male member has been separately removed.

52. A method according to claim 44, wherein the delivering step comprises circulating heated liquid that is heated to about 60°-62° C. external of the subject and directed into the male member of the combination treatment catheter/stent at an inlet temperature of about 62° C. to provide heated liquid in the treatment balloon at above ablation temperatures.

53. A method of administering a therapy to a natural lumen or body cavity of a subject, comprising:
inserting a catheter formed of a first member releasably attached to a second member into the body cavity or lumen of a subject;
delivering a therapy to targeted tissue in the lumen or body cavity of the subject via the catheter such that the therapy is administered via a cooperating relationship between the first and second members;
releasing the first and second members from each other after the delivering step; and
removing the second member from the body after the releasing step independently of the first member such that the first member remains in position in the body of the subject.

54. A method according to claim 53, wherein the therapy comprises a drug therapy.

55. A method according to claim 53, wherein the therapy is a thermal therapy comprising at least one of a hyperthermia or hypothermia treatment.

56. A method according to claim 53, wherein the second member comprises a plurality of fixation balloons which radially expand to securely attach the first member to the second member and which collapse to release the second member from the first member.

57. A method according to claim 55, wherein the thermal therapy is generated locally by a heating element positioned distally in the catheter.

58. A method according to claim 56, wherein the first member comprises an inflatable treatment balloon thereon, wherein the first member includes a wall with an inner surface, wherein the second member includes a wall with an outer surface, and wherein the plurality of fixation balloons define at least two separate fluidly sealed flow chambers between the first and second members.

59. A method according to claim 58, wherein the thermal therapy is generated by directing circulating heated and/or cooled liquid-through the second member such that it exits out of the second member through a selected one of the at least two flow chambers into the first member treatment balloon.

60. A method according to claim 59, wherein the circulating heated liquid exits the treatment balloon to travel through another one of the at least two chambers to return to the second member and exit the body of the subject.

61. A method of making a combination treatment catheter/stent device configured for insertion into a body cavity or lumen of a biological subject, the method comprising:
configuring a flexible elongated tubular body using a first member and a releasably attached second member, wherein, when attached and in position in a subject, said first and second members cooperate to generate and deliver a desired therapy to tissue in a localized region of the body of the subject, and wherein after the desired therapy has been delivered, the first and second members are disengagable from each other while they are held in vivo and a selected one of the members is slidably removable from the body of the subject while the other member remains in position proximate the treated tissue.

62. A method according to claim 61, further comprising:
forming the first member with an inner cavity and an outwardly expandable thermal treatment balloon; and
forming the second member so that it is sized and configured to be received within said first member cavity and provides at least one fluid flow channel, so that when properly attached together, the first and second members are oriented to be in cooperating alignment so as to be able to transfer liquid from the fluid flow channel in the second member to the treatment balloon on the first member to deliver a thermal therapy.

63. A method according to claim 62, wherein the second member is configured to be removable from the first member and is independently removeable from the subject while the first member is adapted to remain in the subject as a post-treatment stent held proximate the operative treatment location for a desired post-treatment duration.

64. A method according to claim 62, wherein the first member is a female member and the second member is a male member.

65. A method according to claim 61, wherein the first member is a male member and the second member is a female member.

66. A method according to claim 61, further comprising attaching a conduit having a cross-sectional area that is substantially less than that the cross-sectional area of the first and/or second member to a selected one of the first and second members, the selected member being the member adapted to remain in the subject after the other member is removed.

67. A method according to claim 66, further comprising forming a plurality of laterally expandable fixation balloons on at least one of the first and second members so as to releaseably secure the first and second members together.

68. A method according to claim 67, further comprising forming a plurality of enclosed fluid flow channels that extend between the first and second members when the first and second members are attached together.
